SEQUENCE LISTING (18 sequences)

SEQ ID NO: 1 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 2 - protein, 295 aa, Homo sapiens
MSDIEEVVEEYEEEEQEEAAVEEEEDWREDEDEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 3 - protein, 288 aa, Homo sapiens
MSDIEEVVEEYEEEEQEEAAVEEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKQAQTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 4 - protein, 285 aa, Homo sapiens
MSDIEEVVEEYEEEEQEEAAVEEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 5 - protein, 282 aa, Homo sapiens
MSDIEEVVEEYEEEEQEEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKAQTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 6 - protein, 298 aa, Homo sapiens
MSDIEEVVEEYEEEEQEEAAVEEEEDWREDEDEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKQAQTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 7 - protein, 255 aa, Homo sapiens
MSDIEEVVEEYEEEEQEEAAVEEEEDWREDEDEQEEAAEEDAEAEAETEETRAEDEEEEEAKEAEDGPMEESKPKPRSFMPNLVPPKIPDGERVDFDERRRAERAEQQRIRNEREKERQNRLAEERARREEEENRRKAEDEARKKKALSNMMHFGGYIQKTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 8 - protein, 161 aa, Homo sapiens
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLDELKIMLQATGETITEDDIEELMKDGDKNNDGRIDYDEFLEFMKGVE

SEQ ID NO: 9 - DNA, 15 bp, Artificial Sequence
  Description of Artificial Sequence Synthetic oligonucleotide
cttacacttccggca

SEQ ID NO: 10 - DNA, 20 bp, Artificial Sequence
  Description of Artificial Sequence Synthetic oligonucleotide
tgagtctcagcatggcggat

SEQ ID NO: 11 - DNA, 20 bp, Artificial Sequence
  Description of Artificial Sequence Synthetic oligonucleotide
gatgatcgatgaggtggacg

SEQ ID NO: 12 - DNA, 121 bp, Artificial Sequence
  Description of Artificial Sequence Synthetic polynucleotide
tgaggatgctgggccagaaccccacccctgaggagctgcaggagatgatcgatgaggtggccgaagacggtgagccccctcctccccaggctccagaagaaccccagctggctgggggctg

SEQ ID NO: 13 - protein, 90 aa, Artificial Sequence
  Description of Artificial Sequence Synthetic polypeptide
  MOD_RES (26)..(26): Phe or Trp
  MOD_RES (61)..(61): Ile or Gln
  MOD_RES (81)..(81): Met or Gln
ASMTDQQAEARAFLSEEMIAEFKAAXDMWDADGGGDISTKELGTVMRMLGQNPTKEELDAXIEEVDEDGSGTIDFEEFLVXMVRQMKEDA

SEQ ID NO: 14 - protein, 89 aa, Artificial Sequence
  Description of Artificial Sequence Synthetic polypeptide
  MOD_RES (27)..(27): Phe or Trp
  MOD_RES (48)..(48): Leu or Gln
  MOD_RES (57)..(57): Leu or Gln
  MOD_RES (61)..(61): Ile or Gln
MDDIYKAAVEQLTEEQKNEFKAAFDIXVLGAEDGCISTKELGKVMRMXGQNPTPEEXQEMXDEVDEDGSGTVDFDEFLVMMVRCMKDDS

SEQ ID NO: 15 - DNA, 36 bp, Artificial Sequence
  Description of Artificial Sequence Synthetic oligonucleotide
gcaggagatgatcgatgaggtggmcgargacggtga

SEQ ID NO: 16 - DNA, 36 bp, Artificial Sequence
  Description of Artificial Sequence Synthetic oligonucleotide
tcaccgtcctcgtccacctcatcgatcatctcctgc

SEQ ID NO: 17 - DNA, 27 bp, Artificial Sequence
  Description of Artificial Sequence Synthetic oligonucleotide
tgaggatgctgggccagaaccccaccc

SEQ ID NO: 18 - DNA, 83 bp, Artificial Sequence
  Description of Artificial Sequence Synthetic oligonucleotide
caggagatgatcgatgaggtggccgaagacggtgagccccctcctccccaggctccagaagaaccccagctggctgggggctg

(19) United States
(12) Patent Application Publication
(10) Pub. No.: US 2024/0189481 A1
(43) Pub. Date: Jun. 13, 2024

(54) NON-CONTRACTILE CARDIOMYOCYTES FOR CARDIAC REPAIR

(71) Applicant: UNIVERSITY OF WASHINGTON, Seattle, WA (US)

(72) Inventors: Charles E. MURRY, Seattle, WA (US); Michael REGNIER, Seattle, WA (US); Hans REINECKE, Seattle, WA (US); Xiulan YANG, Seattle, WA (US); Dasom YOO, Seattle, WA (US)

(73) Assignee: UNIVERSITY OF WASHINGTON, Seattle, WA (US)

(21) Appl. No.: 18/553,941

(22) PCT Filed: Apr. 6, 2022
(86) PCT No.: PCT/US2022/023636

§ 371 Date: Oct. 4, 2023

Related U.S. Application Data
(60) Provisional application No. 63/171,185, filed on Apr. 6, 2021.

(51) Int. Cl. A61L 27/38; A61K 35/34; A61L 27/52; A61P 9/00

(52) U.S. Cl. CPC A61L 27/3826; A61K 35/34; A61L 27/52; A61P 9/00; A61L 2400/06; A61L 2430/20

(57) ABSTRACT

Described herein are compositions and methods relating to the improvement of cardiac function. Various embodiments relate to transplant compositions comprising cardiomyocytes which are engineered to be non-contractile, and to methods of using such cardiomyocytes or transplant compositions to improve cardiac function, e.g., by administering them to cardiac tissue.

### CROSS-REFERENCE TO RELATED APPLICATIONS

This application is a 35 U.S.C. § 371 National Phase Entry Application of International Application No. PCT/US2022/023636 filed Apr. 6, 2022, which designates the U.S. and claims benefit under claims benefit under 35 U.S.C. § 119(e) of U.S. Provisional Application No. 63/171,185 filed Apr. 6, 2021, the contents of which are incorporated herein by reference in their entirety.

### GOVERNMENT SUPPORT

This invention was made with government support under Grant No. RO1 HL146868, awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 17, 2022, is named 034186-191610WOPT_SL.txt and is 22,819 bytes in size.

### TECHNICAL FIELD

The methods and compositions relate to the use of cardiomyocytes for cardiac repair.

### BACKGROUND

Despite major advances in the treatment of heart failure due to systolic impairment, therapeutic approaches have fallen short of addressing the cause of the problem; injury of the mammalian heart leads to irreversible loss of contractile myocardial tissue which is incapable of regeneration. At the turn of the millennium heart failure was widely identified as an emerging epidemic. To date 5.6 million patients in the US alone and 23 million worldwide are suffering from heart failure with 50% dying within 5 years after being diagnosed. Current treatment is limited to ameliorating symptoms and slowing the natural progression of the disease but fails to compensate for the loss of contractile myocardium post-injury.

### SUMMARY

The methods and compositions provided herein are based, in part, on the discovery that grafted cardiomyocytes do not need to contract or can have impaired contraction in order to improve cardiac function. In addition, these studies indicate that electrically active cells having impaired contractile function, or electrically inactive cells having impaired contractile function can provide a benefit upon engraftment, such as improved cardiac function, through e.g., a paracrine mechanism between cells.

Provided herein in one aspect is a transplant composition comprising a non-contractile cardiomyocyte, wherein the non-contractile cardiomyocyte exhibits impaired excitation-contraction coupling. In one embodiment of this aspect and all other aspects provided herein the non-contractile cardiomyocyte is engineered to exhibit impaired excitation-contraction coupling.

In one embodiment of this aspect and all other aspects provided herein, the non-contractile cardiomyocyte is a human non-contractile cardiomyocyte.

In another embodiment of this aspect and all other aspects provided herein, the non-contractile cardiomyocyte exhibits normal action potentials. In another embodiment of this aspect and all other aspects provided herein, the non-contractile cardiomyocyte exhibits action potential dynamics (e.g., magnitude, shape, duration) that are substantially similar to the action potential dynamics of a wild-type cardiomyocyte differentiated in vitro.

In another embodiment of this aspect and all other aspects provided herein, the non-contractile cardiomyocyte exhibits normal calcium transients. In another embodiment of this aspect and all other aspects provided herein, the non-contractile cardiomyocyte exhibits calcium transient dynamics (e.g., magnitude, shape, duration) that are substantially similar to the calcium channel dynamics of a wild-type cardiomyocyte differentiated in vitro.

In another embodiment of this aspect and all other aspects provided herein, the non-contractile cardiomyocyte exhibits impaired function of a thin filament. In another embodiment of this aspect and all other aspects provided herein, the non-contractile cardiomyocyte is engineered to exhibit impaired function of a thin filament, for example, as compared to a wild-type cardiomyocyte differentiated in vitro.

In another embodiment of this aspect and all other aspects provided herein, the impaired function of a thin filament comprises impaired calcium binding to troponin.

In another embodiment of this aspect and all other aspects provided herein, the impaired calcium binding to troponin is a result of knockout or mutation of at least one troponin isoform.

In another embodiment of this aspect and all other aspects provided herein, the non-contractile cardiomyocyte comprises knockout of troponin I (TnI) and/or troponin T (TnT).

In another embodiment of this aspect and all other aspects provided herein, the non-contractile cardiomyocyte comprises a mutation in troponin C that impairs calcium binding to troponin.

In another embodiment of this aspect and all other aspects provided herein, the mutation in troponin C comprises D65A.

In another embodiment of this aspect and all other aspects provided herein, the mutation in troponin C comprises I61Q, and/or L57Q.

In another embodiment of this aspect and all other aspects provided herein, the non-contractile cardiomyocyte exhibits impaired actin-myosin binding.

In another embodiment of this aspect and all other aspects provided herein, the non-contractile cardiomyocyte is engineered to have impaired actin-myosin binding.

In another embodiment of this aspect and all other aspects provided herein, the impaired actin-myosin binding comprises a mutation in or deletion of myosin.

In another embodiment of this aspect and all other aspects provided herein, the composition further comprises a pharmaceutically acceptable carrier.

In another embodiment of this aspect and all other aspects provided herein, the non-contractile cardiomyocyte is derived from in vitro differentiation of a pluripotent stem cell or a cardiac progenitor cell.

In another embodiment of this aspect and all other aspects provided herein, the non-contractile cardiomyocyte retains normal gap junction function when contacted with a cardiomyocyte.

Another aspect provided herein relates to a method for improving cardiac function in a subject in need thereof, the method comprising administering a cardiac transplant composition as described herein to cardiac tissue of a subject in need thereof.

Another aspect provided herein relates to a method for improving cardiac function in a subject in need thereof, the method comprising: administering a non-contractile cardiomyocyte or a composition thereof to a graft site in cardiac tissue of a recipient in need thereof, thereby improving cardiac function in the subject.

In one embodiment of this aspect and all other aspects provided herein, the non-contractile cardiomyocyte exhibits impaired excitation-contraction coupling.

In one embodiment of this aspect and all other aspects provided herein, the non-contractile cardiomyocyte is engineered to exhibit impaired excitation-contraction coupling.

In another embodiment of this aspect and all other aspects provided herein, the non-contractile cardiomyocyte exhibits normal action potentials. In another embodiment of this aspect and all other aspects provided herein, the action potential dynamics (e.g., magnitude, shape, and duration) are substantially similar to the action potential dynamics of a wild-type in vitro-differentiated cardiomyocyte.

In another embodiment of this aspect and all other aspects provided herein, the non-contractile cardiomyocyte exhibits normal calcium transients. In another embodiment of this aspect and all other aspects provided herein, the calcium transient dynamics (e.g., magnitude, shape, and duration) are substantially similar to the calcium transient dynamics to a wild-type in vitro-differentiated cardiomyocyte.

In another embodiment of this aspect and all other aspects provided herein, the non-contractile cardiomyocyte exhibits impaired function of a thin filament.

In another embodiment of this aspect and all other aspects provided herein, the non-contractile cardiomyocyte is engineered to exhibit impaired function of a thin filament.

In another embodiment of this aspect and all other aspects provided herein, the impaired function of a thin filament comprises impaired calcium binding to troponin.

In another embodiment of this aspect and all other aspects provided herein, the impaired calcium binding to troponin is a result of knockout or mutation of at least one troponin isoform.

In another embodiment of this aspect and all other aspects provided herein, the non-contractile cardiomyocyte comprises knockout of troponin I (TnI) and/or troponin T (TnT).

In another embodiment of this aspect and all other aspects provided herein, the non-contractile cardiomyocyte comprises a mutation in troponin C that impairs calcium binding to troponin.

In another embodiment of this aspect and all other aspects provided herein, the mutation in troponin C comprises D65A.

In another embodiment of this aspect and all other aspects provided herein, the non-contractile cardiomyocyte exhibits impaired actin-myosin binding.

In another embodiment of this aspect and all other aspects provided herein, the non-contractile cardiomyocyte is engineered to exhibit impaired actin-myosin binding.

In another embodiment of this aspect and all other aspects provided herein, the impaired actin-myosin binding comprises a mutation in or deletion of myosin.

In another embodiment of this aspect and all other aspects provided herein, the improvement in cardiac function comprises an increase in regional wall motion, fractional shortening, or ejection fraction.

In another embodiment of this aspect and all other aspects provided herein, the subject in need thereof comprises a cardiac disease or disorder.

In another embodiment of this aspect and all other aspects provided herein, the cardiac disease or disorder comprises impaired contractility.

In another embodiment of this aspect and all other aspects provided herein, the cardiac disease or disorder comprises a myocardial infarction, an ischemia/reperfusion injury, a cardiomyopathy or heart failure.

In another embodiment of this aspect and all other aspects provided herein, the risk or incidence of engraftment arrhythmia is reduced compared to a graft comprising wild-type cardiomyocytes (e.g., wild-type in vitro-differentiated cardiomyocytes).

In another embodiment of this aspect and all other aspects provided herein, the method further comprises administering a wild-type cardiomyocyte in combination with the non-contractile cardiomyocyte.

Another aspect provided herein relates to a transplant composition comprising a cardiomyocyte that exhibits attenuated contractility and impaired excitation-contraction coupling.

Another aspect provided herein relates to a transplant composition comprising a cardiomyocyte engineered to have attenuated contractility and exhibit impaired excitation-contraction coupling.

In another embodiment of this aspect and all other aspects provided herein, the cardiomyocyte engineered to have attenuated contractility is human.

In another embodiment of this aspect and all other aspects provided herein, the non-contractile cardiomyocyte exhibits normal action potentials. In another embodiment of this aspect and all other aspects provided herein, the action potential dynamics (e.g., magnitude, shape, and duration) are substantially similar to the action potential dynamics of a wild-type in vitro-differentiated cardiomyocyte.

In another embodiment of this aspect and all other aspects provided herein, the non-contractile cardiomyocyte exhibits normal calcium transients. In another embodiment of this aspect and all other aspects provided herein, the calcium transient dynamics (e.g., magnitude, shape, and duration) are substantially similar to the calcium transient dynamics to a wild-type in vitro-differentiated cardiomyocyte.

In another embodiment of this aspect and all other aspects provided herein, the non-contractile cardiomyocyte exhibits reduced function of a thin filament.

In another embodiment of this aspect and all other aspects provided herein, the non-contractile cardiomyocyte is engineered to exhibit reduced function of a thin filament.

In another embodiment of this aspect and all other aspects provided herein, the reduced function of a thin filament comprises reduced calcium binding to troponin.

In another embodiment of this aspect and all other aspects provided herein, the reduced calcium binding to troponin is a result of a mutation in troponin C that reduces calcium binding to troponin C by at least 20%.

In another embodiment of this aspect and all other aspects provided herein, the mutation in troponin C comprises I61Q and/or L57Q.

In another embodiment of this aspect and all other aspects provided herein, the cardiomyocyte having attenuated contractility comprises reduced actin-myosin binding relative to wild-type.

In another embodiment of this aspect and all other aspects provided herein, the transplant composition further comprises a pharmaceutically acceptable carrier.

In another embodiment of this aspect and all other aspects provided herein, the cardiomyocyte attenuated to have reduced contractility is derived from in vitro differentiation of a pluripotent stem cell or a cardiac progenitor cell.

In another embodiment of this aspect and all other aspects provided herein, the cardiomyocyte attenuated to have reduced contractility retains normal gap junction function when contacted with a cardiomyocyte.

Another aspect provided herein relates to a method for improving cardiac function in a subject in need thereof, the method comprising administering a cardiac transplant composition as described herein to cardiac tissue of a subject in need thereof.

Another aspect relates to a method for improving cardiac function in a subject in need thereof, the method comprising: administering a cardiomyocyte that exhibits attenuated contractility or a composition thereof to a graft site in cardiac tissue of a recipient in need thereof, thereby improving cardiac function in the subject.

Another aspect relates to a method for improving cardiac function in a subject in need thereof, the method comprising: administering a cardiomyocyte engineered to have attenuated contractility or a composition thereof to a graft site in cardiac tissue of a recipient in need thereof, thereby improving cardiac function in the subject.

In one embodiment of this aspect and all other aspects provided herein, the improvement in cardiac function comprises an increase in regional wall motion, fractional shortening, or ejection fraction.

In another embodiment of this aspect and all other aspects provided herein, the subject in need thereof comprises a cardiac disease or disorder.

In another embodiment of this aspect and all other aspects provided herein, the cardiac disease or disorder comprises reduced contractility.

In another embodiment of this aspect and all other aspects provided herein, the cardiac disease or disorder comprises a myocardial infarction, an ischemia/reperfusion injury, a cardiomyopathy or heart failure.

In another embodiment of this aspect and all other aspects provided herein, the risk or incidence of engraftment arrhythmia is reduced compared to a graft comprising wild-type cardiomyocytes.

In another embodiment of this aspect and all other aspects provided herein, the method further comprises administering a wild-type cardiomyocyte in combination with the cardiomyocyte exhibiting or engineered to have attenuated contractility.

In another embodiment of this aspect and all other aspects provided herein, the cardiomyocyte exhibiting or engineered to have attenuated contractility exhibits impaired excitation-contraction coupling.

In another embodiment of this aspect and all other aspects provided herein, the cardiomyocyte having attenuated contractility exhibits normal action potentials. In another embodiment of this aspect and all other aspects provided herein, the action potential dynamics (e.g., magnitude, shape, and duration) are substantially similar to the action potential dynamics of a wild-type in vitro-differentiated cardiomyocyte.

In another embodiment of this aspect and all other aspects provided herein, the cardiomyocyte having attenuated contractility exhibits normal calcium transients. In another embodiment of this aspect and all other aspects provided herein, the calcium transient dynamics (e.g., magnitude, shape, and duration) are substantially similar to the calcium transient dynamics to a wild-type in vitro-differentiated cardiomyocyte.

In another embodiment of this aspect and all other aspects provided herein, the cardiomyocyte having attenuated contractility exhibits reduced function of a thin filament.

In another embodiment of this aspect and all other aspects provided herein, the reduced function of a thin filament comprises reduced binding of calcium to troponin C.

In another embodiment of this aspect and all other aspects provided herein, the cardiomyocyte having attenuated contractility comprises a mutation in troponin C that reduces calcium binding to troponin.

In another embodiment of this aspect and all other aspects provided herein, the mutation in troponin C comprises I61Q and/or L57Q.

In another embodiment of this aspect and all other aspects provided herein, the cardiomyocyte having attenuated contractility exhibits reduced actin-myosin binding.

Another aspect relates to a unit dosage formulation comprising non-contractile cardiomyocytes for administration to cardiac tissue of a subject in need thereof, the unit dosage formulation comprising 1×10³ to 5×10⁹ non-contractile cardiomyocytes in a gel or matrix. Another aspect relates to a unit dosage formulation comprising cardiomyocytes with attenuated contractility for administration to cardiac tissue of a subject in need thereof, the unit dosage formulation comprising 1×10³ to 5×10⁹ attenuated contractility cardiomyocytes in a gel or matrix. In one embodiment of this and all other aspects, the cardiomyocytes are engineered to attenuate contractility.

In another embodiment of this and all other aspects, the cardiomyocyte of the unit dosage formulation is a human cardiomyocyte.

In another embodiment of this and all other aspects, the cardiomyocyte of the unit dosage formulation exhibits normal action potentials. In another embodiment of this and all other aspects, the cardiomyocyte of the unit dosage formulation exhibits normal calcium transients.

In another embodiment of this and all other aspects, the cardiomyocyte of the unit dosage formulation exhibits impaired function of a thin filament.

In another embodiment of this and all other aspects, the cardiomyocyte of the unit dosage formulation is engineered to exhibit impaired function of a thin filament. In another embodiment of this and all other aspects, the impaired function of a thin filament comprises impaired calcium binding to troponin. In another embodiment of this and all other aspects, the impaired calcium binding to troponin is a result of knockout or mutation of at least one troponin isoform.

In another embodiment of this and all other aspects, the cardiomyocyte of the unit dosage formulation comprises knockout of troponin I (TnI) and/or troponin T (TnT).

In another embodiment of this and all other aspects, the cardiomyocyte of the unit dosage formulation comprises a mutation in troponin C that impairs calcium binding to troponin. In another embodiment of this and all other aspects, the mutation comprises D65A mutation of troponin C. In another embodiment of this and all other aspects, the mutation comprises I61Q and/or L57Q mutation of troponin C.

In another embodiment of this and all other aspects, the cardiomyocyte of the unit dosage formulation exhibits impaired actin-myosin binding. In another embodiment of this and all other aspects, the cardiomyocyte of the unit dosage formulation is engineered to have impaired actin-myosin binding. In another embodiment of this and all other aspects, the impaired actin-myosin binding comprises a mutation in or deletion of myosin.

In another embodiment of this and all other aspects, the unit dosage formulation further comprises a pharmaceutically acceptable carrier.

In another embodiment of this and all other aspects, the cardiomyocyte of the unit dosage formulation is derived from in vitro differentiation of a pluripotent stem cell or a cardiac progenitor cell.

In another embodiment of this and all other aspects, the cardiomyocyte of the unit dosage formulation retains normal gap junction function when contacted with a cardiomyocyte.

In another embodiment of this and all other aspects, the gel or matrix comprises a solubilized basement membrane protein or preparation thereof.

In another embodiment of this and all other aspects, the unit dosage formulation further comprises one or more of an immunosuppressive agent, a pan-caspase inhibitor, an anti-apoptotic agent, IGF-1 and a KATP channel opening agent.

In another embodiment of this and all other aspects, the unit dosage formulation comprises 1×10³ to 5×10⁹ non-contractile or attenuated contractility cardiomyocytes. In another embodiment of this and all other aspects, the unit dosage formulation comprises 1×10⁴ to 5×10⁹ non-contractile or attenuated contractility cardiomyocytes. In another embodiment of this and all other aspects, the unit dosage formulation comprises 1×10⁵ to 5×10⁹ non-contractile or attenuated contractility cardiomyocytes. In another embodiment of this and all other aspects, the unit dosage formulation comprises 1×10⁶ to 5×10⁹ non-contractile or attenuated contractility cardiomyocytes. In another embodiment of this and all other aspects, the unit dosage formulation comprises 1×10⁷ to 5×10⁹ non-contractile or attenuated contractility cardiomyocytes. In another embodiment of this and all other aspects, the unit dosage formulation comprises 1×10⁸ to 5×10⁹ non-contractile or attenuated contractility cardiomyocytes. In another embodiment of this and all other aspects, the unit dosage formulation comprises 1×10⁵ to 1×10⁹ non-contractile or attenuated contractility cardiomyocytes. In another embodiment of this and all other aspects, the unit dosage formulation comprises 1×10⁵ to 5×10⁸ non-contractile or attenuated contractility cardiomyocytes. In another embodiment of this and all other aspects, the unit dosage formulation comprises 1×10⁵ to 1×10⁸ non-contractile or attenuated contractility cardiomyocytes. In another embodiment of this and all other aspects, the unit dosage formulation comprises 1×10⁵ to 5×10⁷ non-contractile or attenuated contractility cardiomyocytes. In another embodiment of this and all other aspects, the unit dosage formulation comprises 1×10⁵ to 1×10⁷ non-contractile or attenuated contractility cardiomyocytes. In another embodiment of this and all other aspects, the unit dosage formulation comprises 1×10⁵ to 5×10⁶ non-contractile or attenuated contractility cardiomyocytes. In another embodiment of this and all other aspects, the unit dosage formulation comprises 1×10⁵ to 1×10⁶ non-contractile or attenuated contractility cardiomyocytes.

### BRIEF DESCRIPTION OF THE DRAWINGS

FIG. 1 Protein expression data indicating the absence of slow skeletal (TNNI1) and cardiac troponin I (TNNI3) in the double knockout mouse (TNNI double knockout; TNNI-DKO).

FIG. 2 is a schematic diagram depicting an exemplary method of directed differentiation of human induced pluripotent stem cell-derived cardiomyocytes (hiPSC-cardiomyocytes; hiPSC-CM).

FIG. 3 shows micrographs showing that cardiomyocytes derived from TNNI-DKO hiPSCs have sarcomeres but titin (TTN) knockout hiPSC-CM lack sarcomeres.

FIGS. 4A-4D TNNI-DKO hiPSC-CM have Ca2+ transients that are similar to wild-type Ca2+ transients, but with higher amplitude (A, B) and faster kinetics (C, D).

FIG. 5 shows an exemplary experimental timeline used in the rat hiPSC-CM transplantation studies described herein.

FIG. 6 shows echocardiographic effects of hIPS cell-derived cardiomyocyte grafts on post-infarct ventricular function in rats. Note the progressive decline in function in the sham-injected heart receiving saline vehicle, vs. the equivalent improvement in both the WE and KO hiPSC-CM group. All data are presented as mean±s.e.m. *P<0.05 versus Sham at 3 months.

FIG. 7 shows data indicating cardiac recovery in control treated non-human primates (NHP, saline sham injection), wild-type cardiomyocyte treated NHPs and in an NHP treated with non-contractile cardiomyocytes. Each line on the graph represents a single NHP. These data indicate that there is a comparable loss of ejection fraction in all NHPs post infarction. No spontaneous improvement was observed in NHPs receiving vehicle injection while a robust improvement was observed at 4 weeks in all NHPs receiving contractile cardiomyocytes, with further improvement out to 12 weeks. An equivalent improvement was observed at 4 weeks in the one NHP receiving non-contractile cardiomyocytes, and then minimal improvement from weeks 4-12.

FIG. 8 is a schematic depiction of engineered mutations of Troponin C (TnC) and their effect on contractility. FIG. 8 discloses SEQ ID NOS 13-14, respectively, in order of appearance.

FIG. 9 CRISPR/Cas9 Targeting of TNNC1 in hiPSCs. Schematic indicating an exemplary genome editing strategy used to introduce the D56A mutation in the TNNC1 locus of WT human induced pluripotent stem cells (hiPSCs). The guide RNA (gRNA) and the PAM sequence are shown. The cutting site of Cas9 in the exon 6 of human TNNC1 is indicated by a red arrow. The base A (green square) was replaced with C, resulting in Asp65Ala (GAC>GCC). A silent mutation was introduced in the PAM region to avoid re-cutting by Cas9 (G>A: Glu). The single stranded DNA donor has the desired mutations and homologous arms. FIG. 9 discloses SEQ ID NOS 15-18, respectively, in order of appearance.

FIGS. 10A-10C Cardiomyocytes can form and maintain myofibrils without mechanical contraction. (FIG. 10A) Western blot of Day 14 WTC-CM and cTnC D65A-CM to confirm expression of sarcomere proteins. (FIG. 10B) Representative confocal images of Day 60 WTC-CM and cTnC D65A-CM stained for α-actinin and β-MHC/cTnT. Even without mechanical contraction, cTnC D65A-CMs form and maintain myofibrils. Scale bar=10 μm. (FIG. 10C) Representative structured illumination microscopy images of Day 30 post differentiation WTC-CM and cTnC D65A-CM stained for F-actin. cTnC D65A-CM myofibrils are thinner and less organized with less defined Z-lines. Scale bar=3 μm.

FIGS. 11A-11G Cardiomyocytes with non-functioning cTnC show slower calcium release. (FIG. 11A) Representative traces of calcium transient measurement for Day 60 WTC-CM and cTnC D65A-CM paced at 1 Hz. Calcium transient measurements indicate (FIG. 11B) peak amplitudes are not significantly different between WTC-CM and cTnC D65A-CM at all three frequencies, (FIG. 11C) peak times are significantly longer in cTnC D65A-CMs, both (FIG. 11D) times to 50% peak and (FIG. 11E) times to 90% peak are significantly longer in cTnC D65A-CMs, (FIG. 11F) times to 50% decay are significantly longer in cTnC D65A-CMs paced at 1 Hz and 3 Hz, and (FIG. 11G) times to 90% decay are not significantly different. WTC-CM: n=14 for 1 Hz, n=14 for 2 Hz, n=14 for 3 Hz; cTnC D65A-CM: n=19 for 1 Hz, n=22 for 2 Hz, n=10 for 3 Hz; Data are mean±SEM; significance assessed by Student's t-test and defined by *p<0.05, **p<0.01, ***p<0.001.

FIGS. 12A-12E Replacing cTnC D65A with WT cTnC corrects calcium transients. (FIG. 12A) Representative traces of calcium transient measurements of single cells stained with Fura2-AM for Day 30 cTnC D65A-CM for control and post-transduction with AV-WT cTnC paced at 1 and 2 Hz. Transduced cells were analyzed separately depending on whether they were visibly contracting upon pacing (denoted D30 c_AV_beat vs D30 c_AV_no beat). Calcium transient measurements indicate (FIG. 12B) peak amplitudes significantly decrease and (FIG. 12C) peak time significantly decrease after transduction to express WT cTnC at both frequencies regardless of their contraction state. Both times to 50% peak (FIG. 12D) and 90% peak (FIG. 12E) were significantly shorter for transduced cells paced at 1 Hz, however, there was no difference when paced at 2 Hz. D30 c_no virus: n=19 for 1 Hz and n=25 for 2 Hz, D30 c_AV_beat: n=12 for 1 Hz, n=8 for 2 Hz, D30 c_AV_no beat: n=16 for 1 Hz and n=9 for 2 Hz; mean±SEM; significance assessed by Student's t-test and defined by p<0.05 (*) and p<0.001 (***) when compared with control at each frequency.

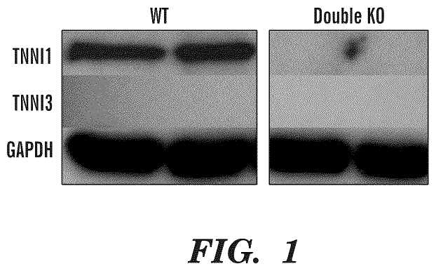

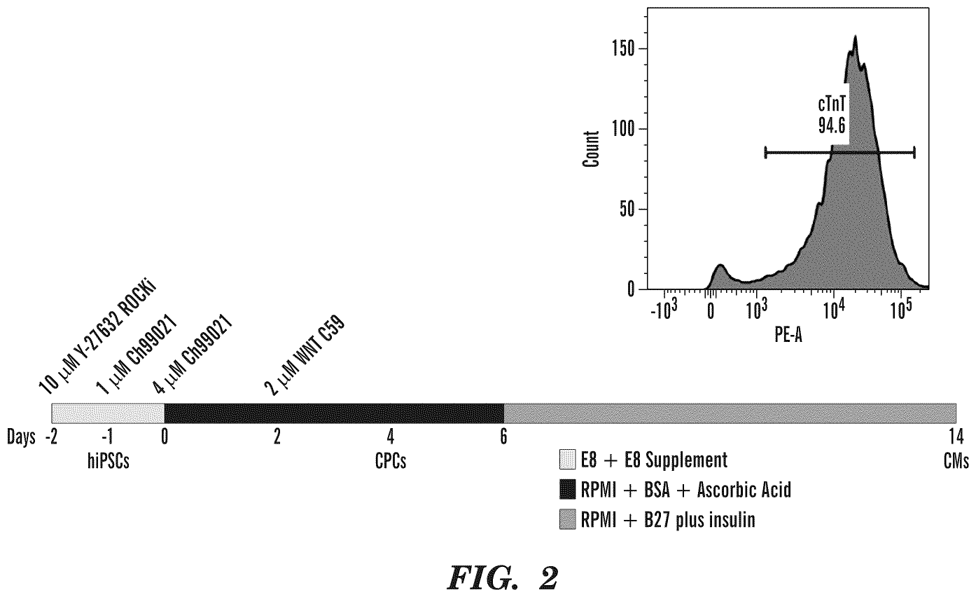

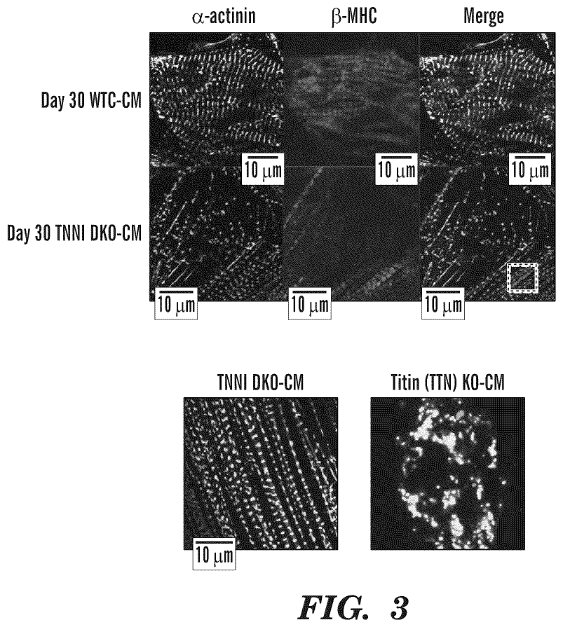

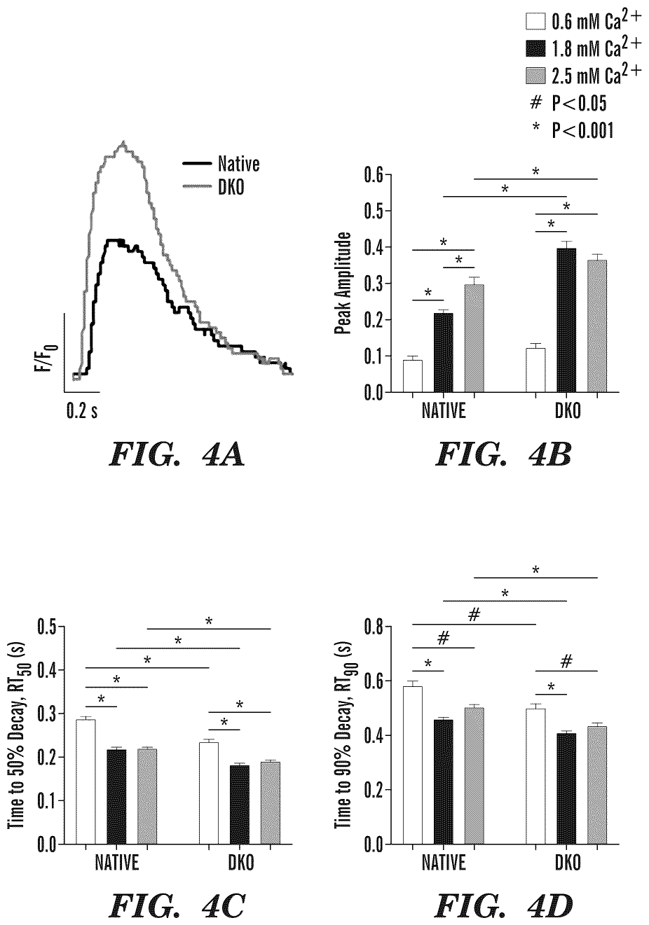

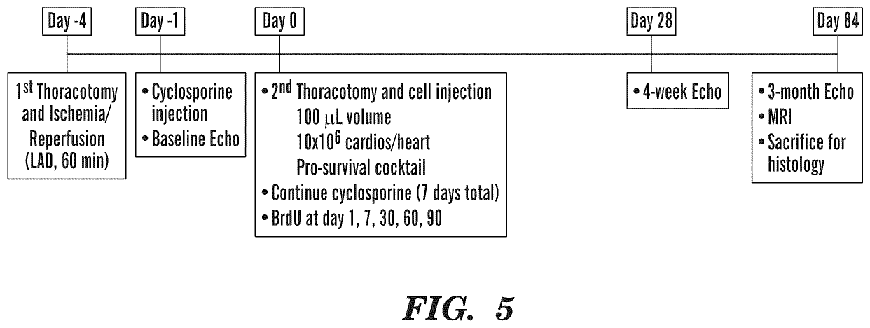

### DETAILED DESCRIPTION

The disclosure described herein is based, in part, on the discovery that cardiomyocytes derived from human pluripotent stem-cells (hPSC) can form stable grafts of new myocardium in injured hearts. These grafts prevent further decline in heart function in small animals (mice, rats, guinea pigs), and restore contractile function in non-human primates, to near-normal levels. A longstanding question in the field is whether cardiomyocyte grafts work directly by adding new force-generating units, or whether passive mechanisms such as mechanically buttressing the infarcted wall or secreting paracrine factors are the predominant mechanism. Prior studies by the inventors have shown that human cardiomyocyte grafts electromechanically couple with the host myocardium and beat in synchrony, providing evidence in support of the direct contractile mechanism. To address this question more directly, human cardiomyocytes that were unable to contract were generated, by knocking out key elements of the calcium-sensing troponin complex (slow skeletal TnI (TNNI1)/cardiac TnI (TNNI3)) in hPSC (TNNI1/3 DKO). These hPSC differentiate normally into cardiomyocytes, form myofibrils with sarcomeres (albeit less robustly than wild type cells), and exhibit rhythmic calcium transients. Importantly, these cells do not beat, indicating that excitation-contraction coupling had been interrupted successfully. These cardiomyocytes were then transplanted into the infarcted hearts of athymic rats and their effects on contractile function were compared to the contractile function of wild type cardiomyocytes. The non-contractile TNNI1/3 DKO cardiomyocytes were equipotent with wild type cardiomyocytes in preventing the decline of systolic function after myocardial infarction. These results indicate that force generation by grafted cardiomyocytes is not necessary to prevent decline in cardiac function post myocardial injury in rats. The ability of non-contractile vs. wild type cardiomyocytes to restore systolic function in infarcted hearts of non-human primates was then compared. For the first 4 weeks post-transplantation, the non-contractile cardiomyocytes were equipotent with wild type cardiomyocytes, restoring ˜10 ejection fraction points, which remained through further time. While wild type cardiomyocytes showed further improvement between weeks 4-12, no further improvement occurred in the heart receiving non-contractile cardiomyocytes, but the improvement seen in the first four weeks was maintained.

### Definitions

As used herein, the term “non-contractile cardiomyocyte” refers to a modified cardiomyocyte in which the excitation properties of the cell are uncoupled from the contraction properties, such that depolarization of the cardiomyocyte plasma membrane does not induce the cell to contract. That is, a non-contractile cardiomyocyte has impaired excitation-contraction coupling. Such non-contractile cardiomyocytes can be genetically modified to be non-contractile, for example, by knockout or mutation of one or more genes or gene products involved in the generation of force by contraction, or conversely, by over-expressing a gene encoding a dominant-negative inhibitor of contraction. Correcting the knockout(s) or gene mutation(s) by viral mediated delivery will restore contractile function of a cardiomyocyte modified to be non-contractile. In some embodiments, the non-contractile cardiomyocyte comprises electrical function that is substantially similar to the electrical function of a wild-type cardiomyocyte. For example, in some embodiments, the non-contractile cardiomyocyte will retain the ability to generate cardiac action potentials and calcium transients that are substantially similar to the action potentials and calcium transients in a wild-type cardiomyocyte. In one embodiment, a non-contractile cardiomyocyte has impaired contractile machinery, for example, one or more impaired contractile myofilaments (e.g., troponin, tropomyosin, myosin) such that the non-contractile cardiomyocyte cannot shorten sarcomere length upon release of Ca2+ from the sarcoplasmic reticulum. As will be appreciated by those of skill in the art, the modification should not adversely affect viability of the cell; mutation of muscle actin at the myosin binding sites to impair actin/myosin interaction could potentially retain the function of actin in the cytoskeleton and not have a negative effect on viability.

As used herein, the term “contractility of the cardiomyocyte or cardiac tissue” refers to the measurement of force of contraction of cardiomyocytes at the cell or tissue level. While contractility can be indirectly assessed by measuring elements of the electrical action of wild-type cardiomyocytes (e.g., cardiac calcium transients), such measurements are not useful where non-contractile cardiomyocytes are involved. Exemplary measures of contractile force include, but are not limited to, sarcomere shortening, and direct contractile force measurement. It will be readily apparent to those of skill in the art that these measures can also be used to detect the absence of contractile activity in non-contractile cardiomyocytes.

As used herein, the term “electrically active cardiomyocytes” refers to non-contractile or wild-type cardiomyocytes that respond to stimuli by local depolarization of the sarcolemmal membrane. Exemplary measurements of electrical activity can include, for example, electrophysiology measurement such as patch clamp and microelectrode arrays, and optical measurement such as fluorescence imaging (e.g., of voltage or calcium transients). For example, optical measurement can be performed using fluorescence dyes specific for different biomolecules, such as calcium dyes (e.g., Fluo-4 AM, Rhod-2 AM).

Conversely, the term “electrically inactive cardiomyocytes” refers to cardiomyocytes that are engineered to remove the ability of the cardiomyocytes to produce an action potential. It is specifically contemplated that the non-contractile cardiomyocytes described herein can also be rendered electrically inactive, while presumably preserving the paracrine function of the cardiomyocytes. Such non-contractile and electrically inactive cardiomyocytes can be used to improve cardiac function while also reducing or preventing the incidence of engraftment arrhythmia in the subject.

As used herein, the term “exhibits normal action potentials” or “exhibits substantially similar action potentials” refers to the ability of a non-contractile cardiomyocyte to produce an electrical action potential that is substantially similar (i.e., less than 25%, 20%, 10%, 5%, 2%, 1% or 0.1% deviation) in magnitude, shape and duration to that of a wild-type cardiomyocyte action potential. Action potential characteristics can be measured using any method known in the art, including but not limited to, microelectrodes, patch clamping (e.g., high throughput or single cell), visualization of voltage transients etc. Similarly, the term ‘exhibits normal calcium transients” or “exhibits substantially similar calcium transients” refers to the ability of a non-contractile cardiomyocyte to produce rhythmic increases in calcium concentration followed by sequestration of calcium and a subsequent reduction in intracellular calcium concentration that is substantially similar (i.e., less than 25%, 20%, 10%, 5%, 2%, 1% or 0.1% deviation) in magnitude, shape and duration to the calcium transients produced in wild-type cardiomyocytes. Calcium transients can be measured using any method known in the art, including but not limited to, fluorescent calcium-sensitive dyes.

As used herein, the term “thin filament” refers to a complex of actin, tropomyosin and troponin; binding of calcium to troponin causes a conformational shift in the position of tropomyosin in the thin filament complex that exposes myosin binding sites on the actin proteins, thereby inducing contraction of the cardiomyocyte. In some embodiments, the thin filament is modified to produce non-contractile cardiomyocytes by disruption of such thin filament function.

As used herein, the phrase “retains normal gap junction activity” refers to the ability of the non-contractile cardiomyocyte to form functional gap junctions with a wild-type cardiomyocyte as assessed using e.g., dual-electrode whole-cell current clamp recordings, or movement of a fluorescent dye from one cell into the next cell. In some embodiments, the normal gap junction activity is at least 75% of the gap junction activity measured between two adjacent wild-type cardiomyocytes; in other embodiments, such normal gap junction activity is at least 80%, at least 85%, at least 90%, at least 92%, at least 95%, at least 98%, at least 99% or even 100% (i.e., substantially similar) of the gap junction activity measured between two wild-type cardiomyocytes.

As used herein, the terms “cardiomyocyte engineered to have attenuated contractility” and “cardiomyocyte engineered to have reduced contractility” are interchangeable and refer to a cardiomyocyte that comprises contractile force that is reduced by at least 10% as compared to the contractile force of a wild-type cardiomyocyte under substantially similar conditions. In other embodiments, the cardiomyocyte engineered to have attenuated contractility comprises a contractile force that is reduced by at least 20%, at least 30%, at least 40%, at least 50%, at least 60%, at least 70%, at least 80%, at least 90%, or at least 95%, or at least 98% compared to the contractile force of a wild-type cardiomyocyte under substantially similar conditions. It is specifically contemplated herein that a cardiomyocyte engineered to have reduced contractility can be used to improve contractility when administered to a graft site in the heart of a subject in need thereof. While a non-contractile cardiomyocyte and a cardiomyocyte engineered to have attenuated contractility are not fully interchangeable, cardiomyocytes with attenuated contractile function can be used in a manner similar to that of the non-contractile cardiomyocytes as described herein.

The term “differentiate”, or differentiating” is a relative term that indicates a “differentiated cell” is a cell that has progressed further down the developmental pathway than its precursor cell. Thus in some embodiments, a stem cell as the term is defined herein, can differentiate to lineage-restricted precursor cells (e.g., a human cardiac progenitor cell or mid-primitive streak cardiogenic mesoderm progenitor cell), which in turn can differentiate into other types of precursor cells further down the pathway (such as a tissue specific precursor, such as a cardiomyocyte progenitor cell), and then to an end-stage differentiated cell (e.g., a cardiomyocyte), which plays a characteristic role in a certain tissue type, and may or may not retain the capacity to proliferate further.

The term “pluripotent” or “pluripotent stem cell (PSC)” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers.

As used herein, the terms “induced pluripotent stem cell,” “iPSC,” “hPSC,” and “human pluripotent stem cell” are used interchangeably herein and refer to a pluripotent cell artificially derived from a differentiated somatic cell (e.g., by reprogramming using one or more methods known in the art). iPSCs are capable of self-renewal and differentiation into cell fate-committed stem cells, including cells of the cardiac lineages, as well as various types of mature cells.

As used herein, “in vitro-differentiated cardiomyocytes” refers to cardiomyocytes that are generated in culture, typically, but not necessarily via step-wise differentiation from a precursor cell such as a human embryonic stem cell, an induced pluripotent stem cell, an early mesoderm cell, a lateral plate mesoderm cell or a cardiac progenitor cell. Thus, while cardiomyocytes in vivo are ultimately derived from a stem cell, i.e., during development of a tissue or organism, a stem cell-derived cardiomyocyte as described herein has been created by in vitro differentiation from a stem cell. Methods for differentiating stem cells in vitro to cardiomyocytes are known in the art and described elsewhere herein. In one embodiment, the cardiomyocytes are differentiated from pluripotent stem cells (e.g., PSC-CMs). In another embodiment, the cardiomyocytes are differentiated directly by reprogramming an adult cell such as a fibroblast. Direct reprogramming can be done either in vitro or in vivo.

As used herein, the term “wild-type cardiomyocyte” refers to a cardiac muscle cell with no genetic modifications to its contractile function that responds to a stimulus by way of depolarization of the sarcoplasmic membrane that induces contraction by inducing sliding of actin thin filaments across myosin thick filaments. Cardiomyocytes generally comprise phenotypic and/or structural features associated with cardiac muscle (e.g., but not limited to, electrical phenotypes, calcium transients, sarcomeres, actin, myosin and cardiac troponin T expression, etc.). In some embodiments, cardiomyocytes are terminally differentiated. In one embodiment, the wild-type cardiomyocyte is an in vitro-differentiated cardiomyocyte.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of cardiomyocytes (contractile or non-contractile), refers to a population of cells that contains fewer than about 20%, more preferably fewer than about 15%, 10%, 8%, 7%, most preferably fewer than about 5%, 4%, 3%, 2%, 1%, or less than 1%, of cells that are not cardiomyocytes, respectively.

The terms “marker” or “cellular marker” as used herein are used to describe a characteristic and/or phenotype of a cell. Markers can be used, for example, for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker. In one embodiment, the marker is a stem cell marker. In one embodiment, the marker is a pluripotent cell marker.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell. As used herein, “iPSC-CMs” or “induced pluripotent stem cell-derived cardiomyocytes” are used interchangeably to refer to cardiomyocytes derived from an induced pluripotent stem cell. Similarly, “PSC-CMs” or “pluripotent stem cell-derived cardiomyocytes” are used interchangeably to refer to cardiomyocytes derived from a pluripotent stem cell. In some embodiments, the terms “hPSC-CM” or “human pluripotent stem cell derived cardiomyocytes” are used interchangeably to refer to cardiomyocytes derived from a human pluripotent stem cell.

As used herein, the terms “transplanting,” “administering” or “engraftment” are used in the context of the placement of cells, e.g., non-contractile cardiomyocytes as described herein into a subject, by a method or route which results in at least partial localization of the introduced cells at a desired site, such as a site of injury or repair, such that a desired effect(s) is produced. The cells e.g., non-contractile cardiomyocytes can be implanted directly to the heart or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of cardiomyocytes is desired as cardiomyocytes do not proliferate to an extent that the heart can heal from an acute injury comprising cell death. Thus, a graft can be used to replace lost cells that occur during injury. A graft can also be used to provide support to cardiac tissue during recovery from a cardiac injury. In other embodiments, the cells can be administered via an indirect systemic route of administration, such as an intraperitoneal or intravenous route. Methods for improving engraftment or preventing engraftment arrhythmias, such as those described in e.g., US 2020-0085880, WO 2020/190739, or WO 2021/163037, the contents of each of which are incorporated herein by reference in their entirety, can be combined with the methods and compositions described herein. In one embodiment, engraftment is used to refer to non-contractile cardiomyocytes that have formed functional gap junctions with endogenous cardiomyocytes in the subject.

As used herein, the term “contacting” when used in reference to a cell, encompasses introducing a cell, agent, surface, scaffold etc. to the cell in a manner that permits physical contact of the cell with the cell, agent, surface, scaffold etc.

As used herein, the term, “cardiac disease” refers to a disease that affects the cardiac tissue of a subject. Non-limiting examples of cardiac diseases include cardiomyopathy, cardiac arrhythmias, myocardial infarction, heart failure, cardiac hypertrophy, long QT syndrome, arrhythmogenic right ventricular dysplasia (ARVD), catecholaminergic polymorphic ventricular tachycardia (CPVT), Barth syndrome, congenital defects, and Duchenne muscular dystrophy.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a disease, or has never received treatment for a disease. A subject can have previously been diagnosed with having a disease, or has never been diagnosed with a disease. A subject can be of any age including, e.g., a fetus, a neonate, a toddler, a child, an adolescent, an adult, a geriatric subject etc.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood other embodiments may include more or less of each element shown in a given Figure. Further, some of the illustrated elements may be combined or omitted. Yet further, an example embodiment may include elements not illustrated in the Figures. As used herein, with respect to measurements, “about” means+/−5%.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

It will be appreciated that, although specific embodiments of the invention have been described herein for purposes of illustration, various modifications may be made without deviating from the spirit and scope of the invention. Accordingly, the invention is not limited except as by the claims.

### Cardiovascular Diseases

The methods described herein can be used to treat, ameliorate, prevent or slow the progression of a number of diseases or their symptoms, such as those resulting in pathological damage to the structure and/or function of the heart (e.g., remodeling in response to heart failure).

A cardiovascular disease is a disease that affects the heart and/or circulatory system of a subject. Such cardiac diseases or cardiac-related disease include, but are not limited to, myocardial infarction, cardiac arrhythmia, heart failure, atherosclerotic heart disease, cardiomyopathy, congenital heart defect (e.g., non-compaction cardiomyopathy, septal defects, hypoplastic left heart), hypertrophic cardiomyopathy, dilated cardiomyopathy, cardiac hypertrophy, myocarditis, arrhythmogenic right ventricular dysplasia (ARVD), long QT syndrome, catecholaminergic polymorphic ventricular tachycardia (CPVT), Barth syndrome, valvular stenosis, regurgitation, ischemia, fibrillation, polymorphic ventricular tachycardia, and muscular dystrophies such as Duchenne or related cardiac disease, and cardiomegaly. Generally, the methods and compositions described herein will be most beneficial for the treatment of cardiac diseases or disorders with impaired contractility, for example, heart failure, myocardial infarction, and cardiomyopathies.

Symptoms of cardiovascular disease can include but are not limited to syncope, fatigue, shortness of breath, chest pain, lower limb edema, and palpitations. A cardiovascular disease is generally diagnosed by a physical examination, blood tests, and/or an electrocardiogram (EKG). An abnormal EKG is an indication that the subject has an abnormal cardiac rhythm or cardiac arrhythmia.

In some embodiments of any of the aspects, the subject has or is at risk for having a cardiovascular disease or a cardiac event.

### Pluripotent Stem Cell Sources

The methods and compositions described herein can be used to generate the non-contractile cardiomyocytes by in vitro differentiation from pluripotent stem cells, e.g., embryonic stem cells, induced pluripotent stem cells, or other stem cells that permit such differentiation and are modified as described herein. The following describes various stem cells that can be used to prepare cardiomyocytes.

Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of stem cells can include amnion-derived or placental-derived stem cells. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Non-contractile cardiomyocytes useful in the methods and compositions described herein can be differentiated from both embryonic stem cells and induced pluripotent stem cells, among others. In one embodiment, the compositions and methods provided herein use human non-contractile cardiomyocytes differentiated from embryonic stem cells. Alternatively, in some embodiments, the compositions and methods provided herein do not encompass generation or use of human cardiogenic cells made from cells taken from a viable human embryo.

Embryonic stem cells and methods for their retrieval are well known in the art and are not described in detail herein. A cell has the phenotype of an embryonic stem cell if it possesses one or more of the unique characteristics of an embryonic stem cell such that that cell can be distinguished from other cells. Exemplary distinguishing embryonic stem cell characteristics include, without limitation, morphology, gene expression or marker profile, proliferative capacity, differentiation capacity, karyotype, responsiveness to particular culture conditions, and the like.

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. The single blastomere cell is co-cultured with established ES-cell lines and then separated from them to form fully competent ES cell lines.

Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli. In some embodiments, the human cardiomyocytes described herein are not derived from embryonic stem cells or any other cells of embryonic origin.

Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g. differences in DNA methylation patterns.

In some embodiments, the methods and compositions described herein utilize non-contractile cardiomyocytes that are differentiated in vitro from induced pluripotent stem cells. An advantage of using iPSCs to generate cardiomyocyte for the compositions described herein is that the cells can be derived from the same subject to which the desired human non-contractile cardiomyocytes are to be administered. That is, a somatic cell can be obtained from a subject, reprogrammed to an induced pluripotent stem cell, and then re-differentiated into a human cardiomyocyte cell to be administered to the subject (e.g., autologous cells). Since the cardiomyocytes (or their differentiated progeny) are essentially derived from an autologous source, the risk of engraftment rejection or allergic responses is reduced compared to the use of cells from another subject or group of subjects.

In some embodiments, the non-contractile cardiomyocytes useful for the compositions described herein are derived from non-autologous or allogeneic sources.

In some embodiments, an iPSC is a cell that has been reprogrammed, a process that alters or reverses the differentiation state of a differentiated cell (e.g., a somatic cell). Stated another way, reprogramming is a process of driving the differentiation of a cell backwards to a more undifferentiated or more primitive type of cell. Reprogramming of somatic cells to induced pluripotent stem cells is known in the art and is not described in detail herein. iPS cells can be generated or derived from terminally differentiated somatic cells, as well as from adult stem cells, or somatic stem cells. That is, a non-pluripotent progenitor cell can be rendered pluripotent or multipotent by reprogramming.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived.

### In Vitro Differentiation of Non-Contractile Cardiomyocytes

The methods and compositions described herein can generate non-contractile cardiomyocytes using in vitro differentiation from cardiac progenitor cells or pluripotent stem cells. Methods for the in vitro differentiation and generation of cardiomyocytes from ESCs or IPSCs are known in the art. See, e.g., Laflamme et al., Nature Biotech 25:1015-1024 (2007) and Nakamura et al, Stem Cell Reports 16:2473-2487 (2021), which describe the differentiation of cardiomyocytes. These approaches use various factors and conditions to activate and guide differentiation programs leading to their respective cell types. Pathways and certain of the factors involved in them are discussed in the following.

In certain embodiments, the step-wise differentiation of ESCs or iPSCs to cardiomyocytes proceeds in the following order: ESC or iPSC>cardiogenic mesoderm>cardiac progenitor cells>cardiomyocytes (see e.g., US 2017024086, the contents of which are incorporated herein by reference in its entirety).

As will be appreciated by those of skill in the art, in vitro-differentiation of cardiomyocytes produces an end-result of a cell having the phenotypic and morphological features of a cardiomyocyte (with the exception of modifications that induce non-contractility as described herein) but that the differentiation steps of in vitro-differentiation need not be the same as the differentiation that occurs naturally in the embryo. That is, during in vitro differentiation to a cardiomyocyte, it is specifically contemplated herein that the step-wise differentiation approach utilized to produce such cells need not proceed through every progenitor cell type that has been identified during embryogenesis and can essentially “skip” over certain stages of development that occur during embryogenesis.

Exemplary methods for generating cardiomyocytes or cardiac progenitor cells from pluripotent stem cells (e.g., embryonic stem cells or induced pluripotent stem cells) are described in e.g., US2020-0085880, the contents of which are incorporated herein by reference in their entirety.

In an alternative approach, cardiac progenitor cells or cardiomyocytes, including non-contractile cardiomyocytes as described herein, can be generated by direct reprogramming or transdifferentiation of an adult somatic cell.

### Engineering Non-Contractile Cardiomyocytes by Disruption of Excitation/Contraction Coupling

The non-contractile cardiomyocytes described herein are engineered to be non-contractile by uncoupling of the excitation/contraction system. While the non-contractile cardiomyocytes can retain electrical activity in the form of action potential propagation and calcium transients, the contractile machinery does not respond to excitation. Essentially any modification to the contractile machinery that renders the cardiomyocyte non-contractile can be used to generate the non-contractile cardiomyocytes described herein, provided that the modification does not impair viability of the cells.

Contraction in cardiac tissue occurs by the sliding of myosin and actin filaments (a sliding filament mechanism) over each other. The energy for this to happen is provided by the hydrolysis of ATP, Myosin functions as an ATPase utilizing ATP to produce a molecular conformational change of part of the myosin and produces movement. Movement of the filaments over each other happens when the globular heads protruding from myosin filaments attach and interact with actin filaments to form crossbridges. The myosin heads tilt and drag along the actin filament a small distance (10-12 nm). The heads then release the actin filament and then changes angle to relocate to another site on the actin filament a further distance (10-12 nm) away. They can then re-bind to the actin molecule and drag it along further. This process is called cross bridge cycling and is the same for all muscles. Crossbridge cycling causes contraction of myosin and actin complexes, in turn causing increased tension along the entire chains of tensile structures, ultimately resulting in contraction of the entire smooth muscle tissue.

Actin-myosin binding is a calcium dependent process. Calcium binds to troponin on the actin thin filament complex, which induces a conformation change in troponin that results in tropomyosin movement that exposes myosin binding sites on the actin chain. Titin is a large protein that binds myosin and attaches it to the z-disc of the sarcomiere.

Modifications that can render cardiomyocytes non-contractile can be made to the components of a thin filament (e.g., troponin, tropomyosin, or actin), a thick filament (e.g., myosin), or other structural components of the actin-myosin system (e.g., titin). A modification can include knockout or mutation of the desired gene. In some embodiments, the non-contractile cardiomyocytes comprise knockout of troponin I (TnI) and/or troponin T (TnT). In other embodiments, the non-contractile cardiomyocytes cause a frame-shift mutation or mutation that causes a truncation of TnI and/or TnT that results in the production of a non-functional TnI and/or TnT. In other embodiments, the modification is a mutation in one or more calcium binding sites of troponin C (e.g., D65A), rendering troponin C unable to bind calcium, thereby inhibiting contraction.

In certain embodiments, the mutation that impairs contractile function in the engineered cardiomyocytes described herein occurs in one or more of the following sequences:

| Troponin I amino acid sequence, human | |
|---|---|
| (SEQ ID NO: 1) | |
| 1 | madgssdaar eprpapapir rrssnyraya tephakkksk isasrklqlk tlllqiakqe |
| 61 | lereaeerrg ekgralstrc qplelaglgf aelqdlcrql harvdkvdee rydieakvtk |
| 121 | niteiadltq kifdlrgkfk rptlrrvris adammqallg arakesldlr ahlkqvkked |
| 181 | tekenrevgd wrknidalsg megrkkkfes |
| Troponin T amino acid sequence, isoform 1, human | |
| (SEQ ID NO: 2) | |
| 1 | msdieevvee yeeeeqeeaa veeeedwred edeqeeaaee daeaeaetee traeedeeee |
| 61 | eakeaedgpm eeskpkprsf mpnlvppkip dgervdfddi hrkrmekdln elqalieahf |
| 121 | enrkkeeeel vslkdrierr raeraeqqri rnerekerqn rlaeerarre eeenrrkaed |
| 181 | earkkkalsn mmhfggyiqk terksgkrqt erekkkkila errkvlaidh lnedqlreka |
| 241 | kelwqsiynl eaekfdlqek fkqqkyeinv lrnrindnqk vsktrgkakv tgrwk |
| Troponin T amino acid sequence, isoform 2, human | |
| (SEQ ID NO: 3) | |
| 1 | msdieevvee yeeeeqeeaa veeqeeaaee daeaeaetee traeedeeee eakeaedgpm |
| 61 | eeskpkprsf mpnlvppkip dgervdfddi hrkrmekdln elqalieahf enrkkeeeel |
| 121 | vslkdrierr raeraeqqri rnerekerqn rlaeerarre eeenrrkaed earkkkalsn |
| 181 | mmhfggyiqk qaqterksgk rqterekkkk ilaerrkvla idhlnedqlr ekakelwqsi |
| 241 | ynleaekfdl qekfkqqkye invlrnrind nqkvsktrgk akvtgrwk |
| Troponin T amino acid sequence, isoform 3, human | |
| (SEQ ID NO: 4) | |
| 1 | msdieevvee yeeeeqeeaa veeqeeaaee daeaeaetee traeedeeee eakeaedgpm |
| 61 | eeskpkprsf mpnlvppkip dgervdfddi hrkrmekdln elqalieahf enrkkeeeel |
| 121 | vslkdrierr raeraeqqri rnerekerqn rlaeerarre eeenrrkaed earkkkalsn |
| 181 | mmhfggyiqk terksgkrqt erekkkkila errkvlaidh lnedqlreka kelwqsiynl |
| 241 | eaekfdlqek fkqqkyeinv lrnrindnqk vsktrgkakv tgrwk |
| Troponin T amino acid sequence, isoform 4, human | |
| (SEQ ID NO: 5) | |
| 1 | msdieevvee yeeeeqeege eaaeedaeae aeteetraee deeeeeakea edgpmeeskp |
| 61 | kprsfmpnlv ppkipdgerv dfddihrkrm ekdlnelgal ieahfenrkk eeeelvslkd |
| 121 | rierrraera eqqrirnere kerqnrlaee rarreeeenr rkaedearkk kalsnmmhfg |
| 181 | gyiqkaqter ksgkrqtere kkkkilaerr kvlaidhlne dqlrekakel wqsiynleae |
| 241 | kfdlqekfkq qkyeinvlrn rindnqkvsk trgkakvtgr wk |
| Troponin T amino acid sequence, isoform 5, human | |
| (SEQ ID NO: 6) | |
| 1 | msdieevvee yeeeeqeeaa veeeedwred edeqeeaaee daeaeaetee traeedeeee |
| 61 | eakeaedgpm eeskpkprsf mpnlvppkip dgervdfddi hrkrmekdln elqalieahf |
| 121 | enrkkeeeel vslkdrierr raeraeqqri rnerekerqn rlaeerarre eeenrrkaed |
| 181 | earkkkalsn mmhfggyiqk qaqterksgk rqterekkkk ilaerrkvla idhlnedqlr |
| 241 | ekakelwqsi ynleaekfdl qekfkqqkye invlrnrind nqkvsktrgk akvtgrwk |
| Troponin T amino acid sequence, isoform 6, human | |
| (SEQ ID NO: 7) | |
| 1 | msdieevvee yeeeeqeeaa veeeedwred edeqeeaaee daeaeaetee traedeeeee |
| 61 | akeaedgpme eskpkprsfm pnlvppkipd gervdfderr raeraeqqri rnerekerqn |
| 121 | rlaeerarre eeenrrkaed earkkkalsn mmhfggyiqk terksgkrqt erekkkkila |
| 181 | errkvlaidh lnedqlreka kelwqsiynl eaekfdlqek fkqqkyeinv lrnrindnqk |
| 241 | vsktrgkakv tgrwk |
| Troponin C amino acid sequence, cardiac and slow skeletal muscle, human | |
| (SEQ ID NO: 8) | |
| 1 | mddiykaave qlteeqknef kaafdifvlg aedgcistke lgkvmrmlgq nptpeelqem |
| 61 | idevdedgsg tvdfdeflvm mvrcmkddsk gkseeelsdl frmfdknadg yidldelkim |
| 121 | lqatgetite ddieelmkdg dknndgridy deflefmkgv e |

The guide RNA (gRNA) sequences used to modify TNNI1 and TNNI3 are as follows:

TNNI1 sgRNA
(SEQ ID NO: 9)
CTTACACTTCCGGCA
TNNI3 sgRNA
(SEQ ID NO: 10)
TGAGTCTCAGCATGGCGGAT

The amino acids that contribute to the calcium binding sites of troponin C are known in the art (see e.g., Li, M et al. Structure and Function of cardiac troponin C (TNNC1I) implications for heart failure, cardiomyopathies, and troponin modulating drugs. Gene 571(2):154-166 (2015)) The crystal structure of troponin C is known—see, e.g., Vassylyev et al., Proc. Nat1. cad. Sci. U.S.A. 95: 4847-4852 (1998), which describes the crystal structure of calcium-bound troponin C in complex with the 47 amino acid N-terminal fragment of TnI. In some embodiments, modification of one of more of the following amino acids can reduce or inhibit calcium binding to troponin C: D29, A31, D33, C35, S37, E40, D65, D67, S69, T71, D73, E176, D105, N107, D109, Y111, D112, E16, D141, N143, D 145, R147, D149, or E152. Certain mutations in troponin C can remove the calcium binding activity entirely, thereby producing a non-contractile cardiomyocyte (e.g., D65A). Alternative mutations in the troponin C calcium binding regions can be used to modulate or “tune” the degree of calcium binding to troponin C. It follows that the degree of contractility of the cardiomyocytes can be engineered to a desired degree to produce cardiomyocytes having attenuated contractility. One of skill will appreciate that mutations in these amino acids should be selected to disrupt calcium binding. The calcium binding site is negatively charged so as to interact with the positively charged calcium ion. Thus modification of amino acids to un-charged/neutral or positively charged (i.e., calcium repelling) amino acids or non-conservative mutations are preferred. Exemplary mutations to modulate troponin C are known in the art and can be used to generate cardionmyocytes having a desired degree of contractility or lack thereof (see e.g., Kreutziger, K L et al. Journal of Molecular and Cellular Cardiology (2011) 50:165-174: Wang, D et al. Archives of Biochemistry and Biophysics (2013) 535:68-75; Powers et al. JCI Insight (2020) 5(20): e142246; Gillis, T E, et al. J Physiol (2007) 561-576; Feest, E R et al Journal of Molecular and Cellular Cardiology (2014) 72:219-227: Davis, J. et al. Cell (2016) 165: 1147-1159, the contents of which are incorporated herein by reference in their entirety.) In one embodiment, the mutation in troponin C comprises I61Q, and/or L57Q. In another embodiment, the mutation in troponin C comprises D65A.

In other embodiments, the mutation can be in the thick filament protein (e.g., myosin). For example, the mutation can disable ATPase activity in the myosin head, thereby impairing normal myosin function of binding actin, myosin head chain power stroke and/or release of myosin from actin.

Mutation of the myosin binding site on actin to prevent actin-myosin interactions is also contemplated herein.

A mutation as described herein can be an amino acid substitution, deletion, frameshift or insertion. It is contemplated herein that a mutation can be any amino acid change that results in the impairment of contractile function or impairment of calcium binding to troponin. In some embodiments, mutation of a small number of amino acid mutations (e.g., 1, 2, 3, 4, or 5) can retain the ability of the targeted protein to properly fold and localize subcellularly with binding partners, but render the protein inert. For example, targeted mutation of TnC permits the formation of a thin filament comprising actin, troponin, and tropomyosin but does not bind calcium and is therefore not responsive to excitation of the cell.

Alterations of the native amino acid sequence (e.g., TnI, TnT, TnC) can be accomplished by any of a number of techniques known in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

In some embodiments, the mutation or knockout of a desired gene involved in cardiac contractility is engineered using a CRISPR/Cas9 system.

In general, “a CRISPR/Cas system” refers collectively to transcripts and other elements involved in the expression of or direction of the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In some embodiments, the CRISPR/Cas system involves a ‘base editing system’ or a ‘prime editing system’ using modified conventional Cas endonucleases to change specific bases without cutting both strands of DNA.

A CRISPR system is typically characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence can comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence.” In aspects of the invention, an exogenous template polynucleotide may be referred to as an editing template. In an aspect of the invention the recombination is homologous recombination.

Methods for CRISPR/Cas mediated genomic modification are known in the art and are not described in detail herein. As non-limiting examples, guide RNA sequences for the mutation of TNNI1, TNNI3 and TnC include the following:

TNNI1 sgRNA:
(SEQ ID NO: 9)
CTTACACTTCCGGCA;
TNNI3 sgRNA:
(SEQ ID NO: 10)
TGAGTCTCAGCATGGCGGAT;
and
TnC sgRNA:
(SEQ ID NO: 11)
GATGATCGATGAGGTGGACG.

A single-stranded donor DNA for use with the TnC sgRNA to introduce the D65A mutation (A->C; plus a silent mutation in the PAM sequence (Glu: GAG->GAA) is:

(SEQ ID NO: 12)
TGAGGATGCTGGGCCAGAACCCCACCCCTGAGGAGCTGCAGGAGATGAT
CGATGAGGTGGCCGAAGACGGTGAGCCCCCTCCTCCCCAGGCTCCAGAA
GAACCCCAGCTGGCTGGGGGCTG.

### Administration of Cells

As used herein, the terms “administering,” “introducing” and “transplanting” are used interchangeably in the context of the placement of cells, e.g. non-contractile cardiomyocytes, as described herein into a subject, by a method or route which results in at least partial localization of the introduced cells at a desired site, such as a site of injury or repair, such that a desired effect(s) is produced. The non-contractile cardiomyocytes can be implanted directly to the heart, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of the non-contractile cardiomyocytes is desired as cardiomyocytes do not proliferate to an extent that the heart can heal from an acute injury comprising cardiomyocyte death.

When provided prophylactically, the non-contractile cardiomyocytes can be administered to a subject in advance of any symptom of a cardiac disorder, e.g., heart failure due to prior myocardial infarction or left ventricular insufficiency, congestive heart failure etc. Accordingly, the prophylactic administration of a population of non-contractile cardiomyocytes serves to prevent a cardiac heart failure disorder or maladaptive cardiac remodeling, as disclosed herein. In some embodiments, the non-contractile cardiomyocytes can be used to improve the function of a normal heart.

The cells described herein can be administered to the heart in an effective amount for the treatment of a cardiac disease or disorder. The term “effective amount” as used herein refers to the amount of a population of non-contractile cardiomyocytes, needed to alleviate at least one or more symptoms of a disease or disorder, including but not limited to a cardiac injury or a cardiac disease or disorder. An “effective amount” relates to a sufficient amount of a composition to provide the desired effect, e.g., treat a subject having an infarct zone following myocardial infarction, improve cardiomyocyte engraftment, prevent onset of heart failure following MI or cardiac injury etc. The term “therapeutically effective amount” therefore refers to an amount of human non-contractile cardiomyocytes, or a composition comprising such cells that is sufficient to promote a particular effect when administered to a typical subject, such as one who has, or is at risk for, a cardiac disease or disorder. An effective amount as used herein would also include an amount sufficient to prevent or delay the development of a symptom of the disease, alter the course of a disease symptom (for example but not limited to, slow the progression of a symptom of the disease), or reverse a symptom of the disease. It is understood that for any given case, an appropriate “effective amount” can be determined by one of ordinary skill in the art using routine experimentation.

In some embodiments, the subject is first diagnosed as having a disease or disorder affecting the myocardium prior to administering the cells according to the methods described herein. In some embodiments, the subject is first diagnosed as being at risk of developing cardiac disease (e.g., heart failure following myocardial injury) or disorder prior to administering the cells.

For use in the various aspects described herein, an effective amount of non-contractile cardiomyocytes comprises at least 1×10³, at least 1×10⁴, at least 1×10⁵, at least 5×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×10⁹ or more non-contractile cardiomyocytes. In the alternative, an effective amount of non-contractile cardiomyocytes comprises at least 1×10³ to 5×10⁹, at least 1×10³ to 1×10⁹, at least 1×10³ to 5×10⁸, at least 1×10³ to 1×10⁸, at least 1×10³ to 5×10⁷, at least 1×10³ to 1×10⁷, at least 1×10³ to 5×10⁶, at least 1×10³ to 1×10⁶, at least 1×10³ to 5×10⁵, at least 1×10³ to 1×10⁵, at least 1×10³ to 5×10⁴, at least 1×10³ to 1×10⁴, at least 1×10⁵ to 5×10⁹, at least 1×10⁵ to 1×10⁹, at least 1×10⁵ to 5×10⁸, at least 1×10⁵ to 1×10⁸, at least 1×10⁵ to 5×10⁷, at least 1×10⁵ to 1×10⁷, at least 1×10⁵ to 5×10⁶, at least 1×10⁵ to 1×10⁶, at least 1×105³ to 5×10⁵, at least 1×10⁶ to 5×10⁹, at least 1×10⁶ to 1×10⁹, at least 1×10⁶ to 5×10⁸, at least 1×10⁶ to 1×10⁸, at least 1×10⁶ to 5×10⁷, at least 1×10⁶ to 1×10⁷, at least 1×10⁶ to 5×10⁶, at least 1×10⁷ to 5×10⁹, at least 1×10⁷ to 1×10⁹, at least 1×10⁷ to 5×10⁸, or at least 1×10⁷ to 1×10⁸ non-contractile cardiomyocytes.

The non-contractile cardiomyocytes can be derived from one or more donors, or can be obtained from an autologous source. In some embodiments of the aspects described herein, the non-contractile cardiomyocytes are expanded or differentiated from progenitor cells in culture prior to administration to a subject in need thereof.

Exemplary modes of administration for use in the methods described herein include, but are not limited to, injection, intracardiac delivery, systemic administration and implantation (with or without a scaffold material). “Injection” includes, without limitation, intracardiac, intravenous, intramuscular, or intraarterial delivery.

In some embodiments, a therapeutically effective amount of non-contractile cardiomyocytes is administered using direct injection into the heart including, but not limited to administration during open-heart surgery, minimally invasive surgery, or by intracardiac injection through an intact chest. These methods are particularly aimed at therapeutic and prophylactic treatments of human subjects having, or at risk of having, a cardiac disease or disorder. The non-contractile cardiomyocytes described herein can be administered to a subject having any cardiac disease or disorder by any appropriate route which results in an effective treatment in the subject. In some embodiments of the aspects described herein, a subject having a cardiac disorder is first selected prior to administration of the cells.

In some embodiments, an effective amount of non-contractile cells is administered to a subject by intracardiac administration or delivery. As defined herein, “intracardiac” administration or delivery refers to all routes of administration whereby a population of non-contractile cardiomyocytes is administered in a way that results in direct contact of these cells with the myocardium of a subject, including, but not limited to, direct cardiac injection, intra-myocardial injection(s), intra-infarct zone injection, injection during surgery (e.g., cardiac bypass surgery, during implantation of a cardiac mini-pump or a pacemaker, etc.). In some such embodiments, the cells are injected into the myocardium (e.g., cardiomyocytes), or into the cavity of the atria and/or ventricles. In some embodiments, intracardiac delivery of cells includes administration methods whereby cells are administered, for example as a cell suspension, to a subject undergoing surgery via a single injection or multiple “mini” injections into the desired region of the heart.

The choice of formulation for a cell composition will depend upon the specific composition used and the number of non-contractile cardiomyocytes to be administered; such formulations can be adjusted by the skilled practitioner. However, as an example, where the composition includes non-contractile cardiomyocytes in a pharmaceutically acceptable carrier, the composition can include a suspension of the cells in an appropriate buffer at an effective concentration of cells per mL of solution. The formulation can also include cell nutrients, a simple sugar (e.g., for osmotic pressure regulation) or other components to maintain the viability and/or assist in delivery and establishment of the cells at the graft site.

U.S. Pat. No. 7,875,451, the entirety of which is incorporated herein by reference, describes formulations that enhance survival of cells transplanted to cardiac tissue. Formulations can include reagents that enhance graft cell survival, promote vascularization by the host, control the fibrotic response, and/or modulate inflammation to promote regeneration. These may be delivered in aqueous media or in natural or synthetic gel compounds. Cell suspension formulations can include, for example, any one or more of a solubilized basement membrane protein or preparation thereof, an immunosuppressive agent, a pan-caspase inhibitor, an anti-apoptotic agent, IGF-1, and a KATP channel opening agent. Alternatively, or in addition, the formulation can comprise a scaffold, such as a biodegradable scaffold.

A matrix, structure, or scaffold can be used to aid in further controlling and directing a cell or population of non-contractile cardiomyocytes or cardiomyocytes engineered to have attenuated contractility as described herein. A matrix or scaffold can be designed or selected to provide environmental cues to control and direct the migration of such cells to a site of cardiac injury or disease. An exemplary matrix for use in preparation of a therapeutic composition comprising cells as described herein is a solubilized basement membrane matrix secreted by Engelbreth-Holm-Swarm mouse sarcoma cells (e.g., Matrigel™) or a synthetic alternative thereof (see e.g., Aisenbrey, E & Murphy, W (2020) Nature Reviews Materials 5:539-551, the contents of which are incorporated herein by reference in their entirety). A structure or scaffold can be engineered from a nanometer to micrometer to millimeter to macroscopic length, and can further comprise or be based on factors such as, but not limited to, material mechanical properties, material solubility, spatial patterning of bioactive compounds, spatial patterning of topological features, soluble bioactive compounds, mechanical perturbation (cyclical or static strain, stress, shear, etc), electrical stimulation, and thermal perturbation.

A scaffold can be in any desired geometric conformation, for example, a flat sheet, a spiral, a cone, a v-like structure and the like. A scaffold can be shaped into, e.g., a heart valve, vessel (tubular), planar construct or any other suitable shape. Such scaffold constructs are known in the art (see, e.g., WO02/035992, U.S. Pat. Nos. 6,479,064, 6,461,628, the contents of which are herein incorporated in their entireties by reference). In some embodiments, after culturing the cells on the scaffold, the scaffold is removed (e.g., bioabsorbed or physically removed), and the cells maintain substantially the same conformation as the scaffold, such that, for example, if the scaffold was spiral shaped, the cells form a 3D-engineered tissue that is spiral shaped.

Biopolymer structures can be generated by providing a transitional polymer on a substrate; depositing a biopolymer on the transitional polymer; shaping the biopolymer into a structure having a selected pattern on the transitional polymer (poly(N-Isopropylacrylamide); and releasing the biopolymer from the transitional polymer with the biopolymer's structure and integrity intact. A biopolymer can be selected from a natural or synthetic extracellular matrix (ECM) protein, growth factor, lipid, fatty acid, steroid, sugar and other biologically active carbohydrates, a biologically derived homopolymer, nucleic acids, hormone, enzyme, pharmaceutical composition, cell surface ligand and receptor, cytoskeletal filament, motor protein, silks, polyprotein (e.g., poly(lysine)) or any combination thereof.

The biopolymers used in the generation of the matrices and scaffolds for the embodiments described herein include, but are not limited to, a) extracellular matrix proteins to direct cell adhesion and function (e.g., collagen, fibronectin, laminin, etc.); (b) growth factors to direct cell function specific to cell type (e.g., nerve growth factor, bone morphogenic proteins, vascular endothelial growth factor, etc.); (c) lipids, fatty acids and steroids (e.g., glycerides, non-glycerides, saturated and unsaturated fatty acids, cholesterol, corticosteroids, sex steroids, etc.); (d) sugars and other biologically active carbohydrates (e.g., monosaccharides, oligosaccharides, sucrose, glucose, glycogen, etc.); (e) combinations of carbohydrates, lipids and/or proteins, such as proteoglycans (protein cores with attached side chains of chondroitin sulfate, dermatan sulfate, heparin, heparan sulfate, and/or keratan sulfate); glycoproteins [e.g., selectins, immunoglobulins, hormones such as human chorionic gonadotropin, Alpha-fetoprotein and Erythropoietin (EPO), etc.]; proteolipids (e.g., N-myristoylated, palmitoylated and prenylated proteins); and glycolipids (e.g., glycoglycerolipids, glycosphingolipids, glycophosphatidylinositols, etc.); (f) biologically derived homopolymers, such as polylactic and polyglycolic acids and poly-L-lysine; (g) nucleic acids (e.g., DNA, RNA, etc.); (h) hormones (e.g., anabolic steroids, sex hormones, insulin, angiotensin, etc.); (i) enzymes (types: oxidoreductases, transferases, hydrolases, lyases, isomerases, ligases; examples: trypsin, collegenases, matrix metallproteinases, etc.); (j) pharmaceuticals (e.g., beta blockers, vasodilators, vasoconstrictors, pain relievers, gene therapy, viral vectors, anti-inflammatories, etc.); (k) cell surface ligands and receptors (e.g., integrins, selectins, cadherins, etc.); (l) cytoskeletal filaments and/or motor proteins (e.g., intermediate filaments, microtubules, actin filaments, dynein, kinesin, myosin, etc.), or any combination thereof. For example, a biopolymer can be selected from the group consisting of fibronectin, vitronectin, laminin, collagen, fibrinogen, silk or silk fibroin.

In some embodiments of the compositions and methods described herein, cardiomyocytes engineered to have modified contractility are encapsulated within permeable membranes prior to implantation. Several methods of cell encapsulation can be employed. In some embodiments, cells will be individually encapsulated. In other instances, many cells will be encapsulated within the same membrane. Several methods of cell encapsulation are well known in the art, such as described in European Patent Publication No. 301,777, or U.S. Pat. Nos. 4,353,888, 4,744,933, 4,749,620, 4,814,274, 5,084,350, and 5,089,272.

In one method of cell encapsulation, the isolated cells are mixed with sodium alginate and extruded into calcium chloride so as to form gel beads or droplets. The gel beads are incubated with a high molecular weight (e.g., MW 60-500 kDa) concentration (0.03-0.1% w/v) polyamino acid (e.g., poly-L-lysine) to form a membrane. The interior of the formed capsule is re-liquified using sodium citrate. This creates a single membrane around the cells that is highly permeable to relatively large molecules (MW ˜200-400 kDa), but retains the cells inside. The capsules are incubated in physiologically compatible carrier for several hours in order that the entrapped sodium alginate diffuses out and the capsules expand to an equilibrium state. The resulting alginate-depleted capsules is reacted with a low molecular weight polyamino acid which reduces the membrane permeability (MW cut-off 40-80 kDa).

Other exemplary materials suitable for use in matrices and scaffolds include, but are not limited to, PEG diacylate, hyaluronic acid, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon, nylon silicon, and shape memory materials, such as poly(styrene-block-butadiene), polynorbornene, hydrogels, metallic alloys, and oligo(-caprolactone)diol as switching segment/oligo(p-dioxyanone)diol as physical crosslink. Other suitable polymers can be obtained by reference to The Polymer Handbook, 3rd edition (Wiley, N.Y., 1989), the contents of which are herein incorporated in their reference by entirety.

In some embodiments, non-contractile cardiomyocytes as described herein can be formulated for administration in a unit dosage formulation. In some embodiments, non-contractile cardiomyocytes as described herein can be administered in a unit dose, as a unit dosage formulation comprising the non-contractile cardiomyocytes. The unit dose in such formulation can include an effective amount of non-contractile cardiomyocytes as described herein. As non-limiting examples, a unit dose formulation can include at least 1×10³, at least 1×10⁴, at least 1×10⁵, at least 5×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×10⁹ or more non-contractile cardiomyocytes. In the alternative, a unit dose formulation can include at least 1×10³ to 5×10⁹, at least 1×10³ to 1×10⁹, at least 1×10³ to 5×10⁸, at least 1×10³ to 1×10⁸, at least 1×10³ to 5×10⁷, at least 1×10³ to 1×10⁷, at least 1×10³ to 5×10⁶, at least 1×10³ to 1×10⁶, at least 1×10³ to 5×10⁵, at least 1×10³ to 1×10⁵, at least 1×10³ to 5×10⁴, at least 1×10³ to 1×10⁴, at least 1×10⁵ to 5×10⁹, at least 1×10⁵ to 1×10⁹, at least 1×10⁵ to 5×10⁸, at least 1×10⁵ to 1×10⁸, at least 1×10⁵ to 5×10⁷, at least 1×10⁵ to 1×10⁷, at least 1×10⁵ to 5×10⁶, at least 1×10⁵ to 1×10⁶, at least 1×105³ to 5×10⁵, at least 1×10⁶ to 5×10⁹, at least 1×10⁶ to 1×10⁹, at least 1×10⁶ to 5×10⁸, at least 1×10⁶ to 1×10⁸, at least 1×10⁶ to 5×10⁷, at least 1×10⁶ to 1×10⁷, at least 1×10⁶ to 5×10⁶, at least 1×10⁷ to 5×10⁹, at least 1×10⁷ to 1×10⁹, at least 1×10⁷ to 5×10⁸, or at least 1×10⁷ to 1×10⁸ non-contractile cardiomyocytes.

In some embodiments, the unit dosage formulation comprises a gel or matrix with the non-contractile cardiomyocytes suspended therein. In some embodiments, the gel or matrix comprises a solubilized basement membrane protein or preparation thereof. Unit dosage formulations can include reagents that enhance graft cell survival, promote vascularization by the host, control the fibrotic response, and/or modulate inflammation to promote regeneration. As noted above, formulations that enhance survival of cells transplanted to cardiac tissue are described, for example, in U.S. Pat. No. 7,875,451, which is incorporated herein by reference in its entirety. Non-limiting examples of agents that enhance graft survival can include any one or more of an immunosuppressive agent, a pan-caspase inhibitor, an anti-apoptotic agent, IGF-1 and a KATP channel opening agent.

In some embodiments, additional agents to aid in treatment of the subject can be administered before or following treatment with the cells as described herein. Such additional agents can be used to prepare the target tissue for administration of the cells. Alternatively, the additional agents can be administered after the cell transplantation to support the engraftment and growth of the administered cells into the heart. In some embodiments, the additional agent comprises growth factors, such as VEGF or PDGF. Other exemplary agents can be used to reduce the load on the heart while the non-contractile cardiomyocytes are engrafting (e.g., beta blockers, medications to lower blood pressure etc.).

In some embodiments, it is specifically contemplated herein that non-contractile cardiomyocytes are administered in combination with wild-type (i.e., contractile) cardiomyocytes. Such co-administration can result in improved engraftment of the contractile cardiomyocytes into cardiac tissue, for example, by a paracrine mechanism that can promote cell viability and/or alterations to the extracellular matrix. In one embodiments, the ratio of non-contractile cardiomyocytes to wild-type cardiomyocytes is 1:1. In other embodiments, the ratio of non-contractile cardiomyocytes to wild-type cardiomyocytes is 1:2, 1:3, 1:4, 1:5, 1:10, 1:20, 1:25, 1:50, 1:100, 2:1, 3:1, 4:1, 5:1, 10:1, 20:1, 25:1, 50:1 100:1 or any ratio therebetween.

### Efficacy

The efficacy of a given treatment for improving cardiac function, e.g., improving fractional shortening, ejection fraction or other measures of cardiac contractility (e.g., whole heart function), in vivo can be determined by the skilled clinician. However, a treatment is considered “effective treatment,” as the term is used herein, if any one or all of the signs or symptoms of impaired cardiac contractility is/are altered in a beneficial manner, or other clinically accepted symptoms or markers of disease are improved, or ameliorated, e.g., by at least 10% following treatment with e.g., a composition comprising non-contractile cardiomyocytes. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease or (2) relieving the disease, e.g., causing regression of symptoms; and (3) preventing or reducing the likelihood of the development of the disease, or preventing secondary diseases/disorders (e.g., cellular edema, pneumonia associated with heart failure etc).

An effective amount for the treatment of a disease means that amount which, when administered to a mammal in need thereof, is sufficient to result in effective treatment as that term is defined herein, for that disease Efficacy of an agent can be determined by assessing a reduction in one or more physical indicators of the disease, such as e.g., lung congestion, edema, shortness of breath, exercise intolerance, rapid heart rate, chest pain, fainting etc. For use in the various aspects described herein, an effective amount of non-contractile cardiomyocytes for the treatment of disease comprises at least 1×10³, at least 1×10⁴, at least 1×10⁵, at least 5×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×10⁹ or more non-contractile cardiomyocytes. In the alternative, an effective amount of non-contractile cardiomyocytes for the treatment of disease comprises at least 1×10³ to 5×10⁹, at least 1×10³ to 1×10⁹, at least 1×10³ to 5×10⁸, at least 1×10³ to 1×10⁸, at least 1×10³ to 5×10⁷, at least 1×10³ to 1×10⁷, at least 1×10³ to 5×10⁶, at least 1×10³ to 1×10⁶, at least 1×10³ to 5×10⁵, at least 1×10³ to 1×10⁵, at least 1×10³ to 5×10⁴, at least 1×10³ to 1×10⁴, at least 1×10⁵ to 5×10⁹, at least 1×10⁵ to 1×10⁹, at least 1×10⁵ to 5×10⁸, at least 1×10⁵ to 1×10⁸, at least 1×10⁵ to 5×10⁷, at least 1×10⁵ to 1×10⁷, at least 1×10⁵ to 5×10⁶, at least 1×10⁵ to 1×10⁶, at least 1×105³ to 5×10⁵, at least 1×10⁶ to 5×10⁹, at least 1×10⁶ to 1×10⁹, at least 1×10⁶ to 5×10⁸, at least 1×10⁶ to 1×10⁸, at least 1×10⁶ to 5×10⁷, at least 1×10⁶ to 1×10⁷, at least 1×10⁶ to 5×10⁶, at least 1×10⁷ to 5×10⁹, at least 1×10⁷ to 1×10⁹, at least 1×10⁷ to 5×10⁸, or at least 1×10⁷ to 1×10⁸ non-contractile cardiomyocytes.

Exemplary indicators of cardiac disease or cardiac disorder, or cardiac injury can be monitored to determine the efficacy of treatment. Non-limiting examples of such functional indicators or parameters, e.g., stroke volume, heart rate, cardiac output, left ventricular ejection fraction, heart rhythm, blood pressure, heart volume, regurgitation, etc. as well as biochemical indicators, such as a decrease in markers of cardiac injury, such as serum lactate dehydrogenase, or serum troponin, among others, or by reductions in blood markers of heart failure, e.g., BNP or ANP. As one example, myocardial ischemia and reperfusion are associated with reduced cardiac function. Subjects that have suffered an ischemic cardiac event and/or that have received reperfusion therapy have reduced cardiac function when compared to that before ischemia and/or reperfusion. Measures of cardiac function include, for example, ejection fraction and fractional shortening. Ejection fraction is the fraction of blood pumped out of a ventricle with each heartbeat and the term applies to both the right and left ventricles. LVEF refers to the left ventricular ejection fraction (LVEF). Fractional shortening refers to the difference between end-diastolic and end-systolic dimensions divided by end-diastolic dimension. Typically, the left ventricular ejection fraction of a normal heart ranges between 55%-70% while that of a failing heart can be less than 35%. In some embodiments, administration of non-contractile cardiomyocytes or cardiomyocytes engineered to have reduced contractility to cardiac tissue of a subject (e.g., a subject having reduced left ventricular ejection fraction) increases the LVEF of the heart by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45% or more.

Non-limiting examples of clinical tests that can be used to assess cardiac functional parameters include echocardiography (with or without Doppler flow imaging), electrocardiogram (EKG), exercise stress test, Holter monitoring, or measurement of β-natriuretic peptide.

Where necessary or desired, animal models of cardiac injury or cardiac disease can be used to gauge the effectiveness of a particular composition as described herein. For example, an isolated working rabbit or rat heart model, or a coronary ligation model in either canines or porcines can be used. Animal models of cardiac function are useful for monitoring infarct zones, coronary perfusion, electrical conduction, left ventricular end diastolic pressure, left ventricular ejection fraction, heart rate, blood pressure, degree of hypertrophy, diastolic relaxation function, cardiac output, heart rate variability, and ventricular wall thickness, etc.

The invention may be as described in any one of the following numbered paragraphs:

1. A transplant composition comprising a non-contractile cardiomyocyte, wherein the non-contractile cardiomyocyte exhibits impaired excitation-contraction coupling.

2. The transplant composition of paragraph 1, wherein the non-contractile cardiomyocyte is engineered to exhibit impaired excitation-contraction coupling.

3. The transplant composition of paragraph 1 or paragraph 2, wherein the non-contractile cardiomyocyte is a human non-contractile cardiomyocyte.

4. The transplant composition of any preceding paragraph, wherein the non-contractile cardiomyocyte exhibits normal action potentials.

5. The transplant composition of any preceding paragraph, wherein the non-contractile cardiomyocyte exhibits normal calcium transients.

6. The transplant composition of any preceding paragraph, wherein the non-contractile cardiomyocyte exhibits impaired function of a thin filament.

7. The transplant composition of any preceding paragraph, wherein the non-contractile cardiomyocyte is engineered to exhibit impaired function of a thin filament.

8. The transplant composition of any preceding paragraph, wherein impaired function of a thin filament comprises impaired calcium binding to troponin.

9. The transplant composition of any preceding paragraph, wherein impaired calcium binding to troponin is a result of knockout or mutation of at least one troponin isoform.

10. The transplant composition of any preceding paragraph, wherein the non-contractile cardiomyocyte comprises knockout of troponin I (TnI) and/or troponin T (TnT).

11. The transplant composition of any preceding paragraph, wherein the non-contractile cardiomyocyte comprises a mutation in troponin C that impairs calcium binding to troponin.

12. The transplant composition of paragraph 11, wherein the mutation in troponin C comprises D65A.

13. The transplant composition of paragraph 11, wherein the mutation in troponin C comprises I61Q, and/or L57Q.

14. The transplant composition of any preceding paragraph, wherein the non-contractile cardiomyocyte exhibits impaired actin-myosin binding.

15. The transplant composition of any preceding paragraph, wherein the non-contractile cardiomyocyte is engineered to have impaired actin-myosin binding.

16. The transplant composition of paragraph 14 or 15, wherein the impaired actin-myosin binding comprises a mutation in or deletion of myosin.

17. The transplant composition of any preceding paragraph, further comprising a pharmaceutically acceptable carrier.

18. The transplant composition of any preceding paragraph, wherein the non-contractile cardiomyocyte is derived from in vitro differentiation of a pluripotent stem cell or a cardiac progenitor cell.

19. The transplant composition of any preceding paragraph, wherein the non-contractile cardiomyocyte retains normal gap junction function when contacted with a cardiomyocyte.

20. A method for improving cardiac function in a subject in need thereof, the method comprising administering a cardiac transplant composition of any one of paragraphs 1-19 to cardiac tissue of a subject in need thereof.

21. A method for improving cardiac function in a subject in need thereof, the method comprising: administering a non-contractile cardiomyocyte or a composition thereof to a graft site in cardiac tissue of a recipient in need thereof, thereby improving cardiac function in the subject.

22. The method of paragraph 21, wherein the non-contractile cardiomyocyte exhibits impaired excitation-contraction coupling.

23. The method of paragraph 21 or 22, wherein the non-contractile cardiomyocyte is engineered to exhibit impaired excitation-contraction coupling.

24. The method of any one of paragraphs 21-23, wherein the non-contractile cardiomyocyte exhibits normal action potentials.

25. The method of claim any one of paragraphs 21-24, wherein the non-contractile cardiomyocyte exhibits normal calcium transients.

26. The method of any one of paragraphs 21-25, wherein the non-contractile cardiomyocyte exhibits impaired function of a thin filament.

27. The method of any one of paragraphs 21-26, wherein the non-contractile cardiomyocyte is engineered to exhibit impaired function of a thin filament.

28. The method of paragraph 26 or 27, wherein the impaired function of a thin filament comprises impaired calcium binding to troponin.

29. The method of paragraph 28, wherein the impaired calcium binding to troponin is a result of knockout or mutation of at least one troponin isoform.

30. The method of any one of paragraphs 22-29, wherein the non-contractile cardiomyocyte comprises knockout of troponin I (TnI) and/or troponin T (TnT).

31. The method of paragraph 28 or 29, wherein the non-contractile cardiomyocyte comprises a mutation in troponin C that impairs calcium binding to troponin.

32. The method of paragraph 31, wherein the mutation in troponin C comprises D65A.

33. The method of any one of paragraphs 22-27, wherein the non-contractile cardiomyocyte exhibits impaired actin-myosin binding.

34. The method of any one of paragraphs 22-27, wherein the non-contractile cardiomyocyte is engineered to exhibit impaired actin-myosin binding.

35. The method of paragraph 33 or 34, wherein the impaired actin-myosin binding comprises a mutation in or deletion of myosin.

36. The method of any one of paragraphs 20-35, wherein the improvement in cardiac function comprises an increase in regional wall motion, fractional shortening, or ejection fraction.

37. The method of any one of paragraphs 20-36, wherein the subject in need thereof comprises a cardiac disease or disorder.

38. The method of paragraph 37, wherein the cardiac disease or disorder comprises impaired contractility.

39. The method of paragraph 37 or 38, wherein the cardiac disease or disorder comprises a myocardial infarction, an ischemia/reperfusion injury, a cardiomyopathy or heart failure.

40. The method of any one of paragraphs 20-39, wherein the risk or incidence of engraftment arrhythmia is reduced compared to a graft comprising wild-type cardiomyocytes.

41. The method of any one of paragraphs 20-40, further comprising administering a wild-type cardiomyocyte in combination with the non-contractile cardiomyocyte.

42. A transplant composition comprising a cardiomyocyte that exhibits attenuated contractility and impaired excitation-contraction coupling.

43. A transplant composition comprising a cardiomyocyte engineered to have attenuated contractility and/or impaired excitation-contraction coupling.

44. The transplant composition of paragraph 42 or 43, wherein the cardiomyocyte is human.

45. The transplant composition of any one of paragraphs 42-44, wherein the cardiomyocyte engineered to have attenuated contractility exhibits normal action potentials.

46. The transplant composition of any one of paragraphs 42-45, wherein the cardiomyocyte engineered to have attenuated contractility exhibits normal calcium transients.

47. The transplant composition of any one of paragraphs 42-46, wherein the non-contractile cardiomyocyte exhibits reduced function of a thin filament.

48. The transplant composition of any one of paragraphs 42-47, wherein the non-contractile cardiomyocyte is engineered to exhibit reduced function of a thin filament.

49. The transplant composition of paragraph 47 or 48, wherein the reduced function of a thin filament comprises reduced calcium binding to troponin.

50. The transplant composition of paragraph 49, wherein the reduced calcium binding to troponin is a result of a mutation in troponin C that reduces calcium binding to troponin C by at least 20%.

51. The transplant composition of paragraph 50, wherein the mutation in troponin C comprises I61Q and/or L57Q.

52. The transplant composition of any one of paragraphs 42-48, wherein the cardiomyocyte comprises reduced actin-myosin binding relative to wild-type.

53. The transplant composition of any one of paragraphs 42-52, further comprising a pharmaceutically acceptable carrier.

54. The transplant composition of any one of paragraphs 42-53, wherein the cardiomyocyte that has reduced contractility is derived from in vitro differentiation of a pluripotent stem cell or a cardiac progenitor cell.

55. The transplant composition of any one of paragraphs 42-54, wherein the cardiomyocyte having reduced contractility retains normal gap junction function when contacted with a cardiomyocyte.

56. A method for improving cardiac function in a subject in need thereof, the method comprising administering a transplant composition of any one of paragraphs 42-55 to cardiac tissue of a subject in need thereof.

57. A method for improving cardiac function in a subject in need thereof, the method comprising: administering a cardiomyocyte that exhibits attenuated contractility or a composition thereof to a graft site in cardiac tissue of the subject in need thereof, thereby improving cardiac function in the subject.

58. The method of paragraph 56 or 57, wherein the improvement in cardiac function comprises an increase in regional wall motion, fractional shortening, or ejection fraction.

59. The method of claim any one of paragraphs 56-58, wherein the subject in need thereof comprises a cardiac disease or disorder.

60. The method of paragraph 59, wherein the cardiac disease or disorder comprises reduced contractility.

61. The method of paragraph 60, wherein the cardiac disease or disorder comprises a myocardial infarction, an ischemia/reperfusion injury, a cardiomyopathy or heart failure.

62. The method of any one of paragraphs 56-61, wherein the risk or incidence of engraftment arrhythmia is reduced compared to a graft comprising wild-type cardiomyocytes.

63. The method of any one of paragraphs 56-61, further comprising administering a wild-type cardiomyocyte in combination with the cardiomyocyte engineered to have attenuated contractility.

64. A unit dosage formulation comprising non-contractile cardiomyocytes for administration to cardiac tissue of a subject in need thereof, the unit dosage formulation comprising 1×10³ to 5×10⁹ non-contractile cardiomyocytes in a gel or matrix.

65. The unit dosage formulation of paragraph 64, wherein the non-contractile cardiomyocyte is engineered to exhibit impaired excitation-contraction coupling.

66. The unit dosage formulation of claim paragraph or claim paragraph, wherein the non-contractile cardiomyocyte is a human non-contractile cardiomyocyte.

67. The unit dosage formulation of any one of paragraphs 64-66, wherein the non-contractile cardiomyocyte exhibits normal action potentials.

68. The unit dosage formulation of any one of paragraphs 64-67, wherein the non-contractile cardiomyocyte exhibits normal calcium transients.

69. The unit dosage formulation of any one of paragraphs 64-68, wherein the non-contractile cardiomyocyte exhibits impaired function of a thin filament.

70. The unit dosage formulation of paragraph 69, wherein the non-contractile cardiomyocyte is engineered to exhibit impaired function of a thin filament.

71. The unit dosage formulation of paragraph 69 or 70, wherein impaired function of a thin filament comprises impaired calcium binding to troponin.

72. The unit dosage formulation of paragraph 71, wherein calcium binding to troponin is a result of knockout or mutation of at least one troponin isoform.

73. The unit dosage formulation of paragraph 72, wherein the non-contractile cardiomyocyte comprises knockout of troponin I (TnI) and/or troponin T (TnT).

74. The unit dosage formulation of paragraph 72, wherein the non-contractile cardiomyocyte comprises a mutation in troponin C that impairs calcium binding to troponin.

75. The unit dosage formulation of paragraph 72 or 74 wherein the mutation comprises D65A mutation of troponin C.

76. The unit dosage formulation of paragraph 72 or 74, wherein the mutation comprises I61Q and/or L57Q mutation of troponin C.

77. The unit dosage formulation of any one of paragraphs 64-76, wherein the non-contractile cardiomyocyte exhibits impaired actin-myosin binding.

78. The unit dosage formulation of any one of paragraphs 64-77, wherein the non-contractile cardiomyocyte is engineered to have impaired actin-myosin binding

79. The unit dosage formulation of paragraph 77 or 78, wherein the impaired actin-myosin binding comprises a mutation in or deletion of myosin.

80. The unit dosage formulation of any one of paragraphs 64-79, further comprising a pharmaceutically acceptable carrier.

81. The unit dosage formulation of any one of paragraphs 64-80, wherein the non-contractile cardiomyocyte is derived from in vitro differentiation of a pluripotent stem cell or a cardiac progenitor cell.

82. The unit dosage formulation of any one of paragraphs 64-81, wherein the non-contractile cardiomyocyte retains normal gap junction function when contacted with a cardiomyocyte.

83. The unit dosage formulation of any one of paragraphs 64-82, wherein the gel or matrix comprises a solubilized basement membrane protein or preparation thereof.

84. The unit dosage formulation of any one of paragraphs 64-83, wherein the formulation further comprises one or more of an immunosuppressive agent, a pan-caspase inhibitor, an anti-apoptotic agent, IGF-1 and a KATP channel opening agent.

85. The unit dosage formulation of any one of paragraphs 64-84, wherein the unit dosage formulation comprises 1×10³ to 5×10⁹ non-contractile cardiomyocytes.

86. The unit dosage formulation of any one of paragraphs 64-85, wherein the unit dosage formulation comprises 1×10⁴ to 5×10⁹ non-contractile cardiomyocytes.

87. The unit dosage formulation of any one of paragraphs 64-86, wherein the unit dosage formulation comprises 1×10⁵ to 5×10⁹ non-contractile cardiomyocytes.

88. The unit dosage formulation of any one of paragraphs 64-68, wherein the unit dosage formulation comprises 1×10⁶ to 5×10⁹ non-contractile cardiomyocytes.

89. The unit dosage formulation of any one of paragraphs 64-88, wherein the unit dosage formulation comprises 1×10⁷ to 5×10⁹ non-contractile cardiomyocytes.

90. The unit dosage formulation of any one of paragraphs 64-89, wherein the unit dosage formulation comprises 1×10⁸ to 5×10⁹ non-contractile cardiomyocytes.

91. The unit dosage formulation of any one of paragraphs 64-90, wherein the unit dosage formulation comprises 1×10⁵ to 1×10⁹ non-contractile cardiomyocytes.

92. The unit dosage formulation of any one of paragraphs 64-91, wherein the unit dosage formulation comprises 1×10⁵ to 5×10⁸ non-contractile cardiomyocytes.

93. The unit dosage formulation of any one of paragraphs 64-92, wherein the unit dosage formulation comprises 1×10⁵ to 1×10⁸ non-contractile cardiomyocytes.

94. The unit dosage formulation of any one of paragraphs 64-93, wherein the unit dosage formulation comprises 1×10⁵ to 5×10⁷ non-contractile cardiomyocytes.

95. The unit dosage formulation of any one of paragraphs 64-94, wherein the unit dosage formulation comprises 1×10⁵ to 1×10⁷ non-contractile cardiomyocytes.

96. The unit dosage formulation of any one of paragraphs 64-95, wherein the unit dosage formulation comprises 1×10⁵ to 5×10⁶ non-contractile cardiomyocytes.

97. The unit dosage formulation of any one of paragraphs 66-96, wherein the unit dosage formulation comprises 1×10⁵ to 1×10⁶ non-contractile cardiomyocytes.

The invention is further described in the following examples, which do not limit the scope of the invention described in the claims.

### Examples

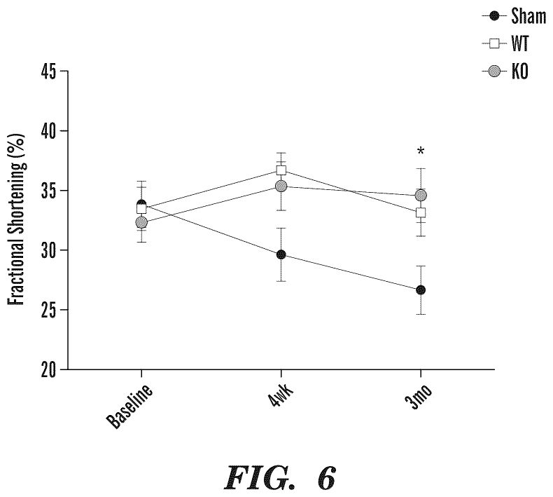

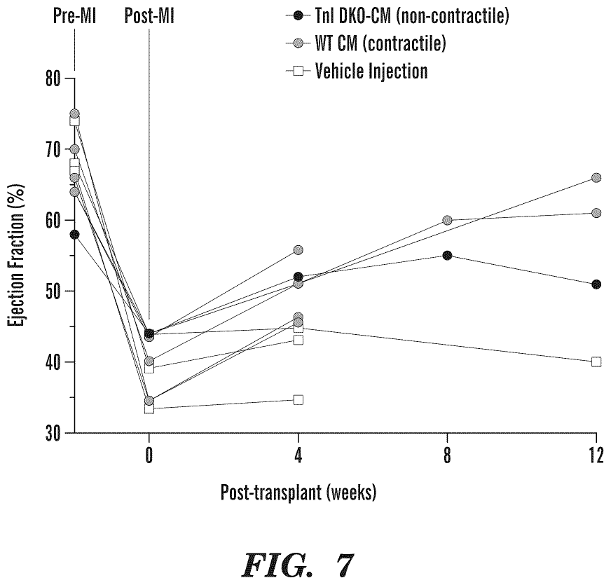

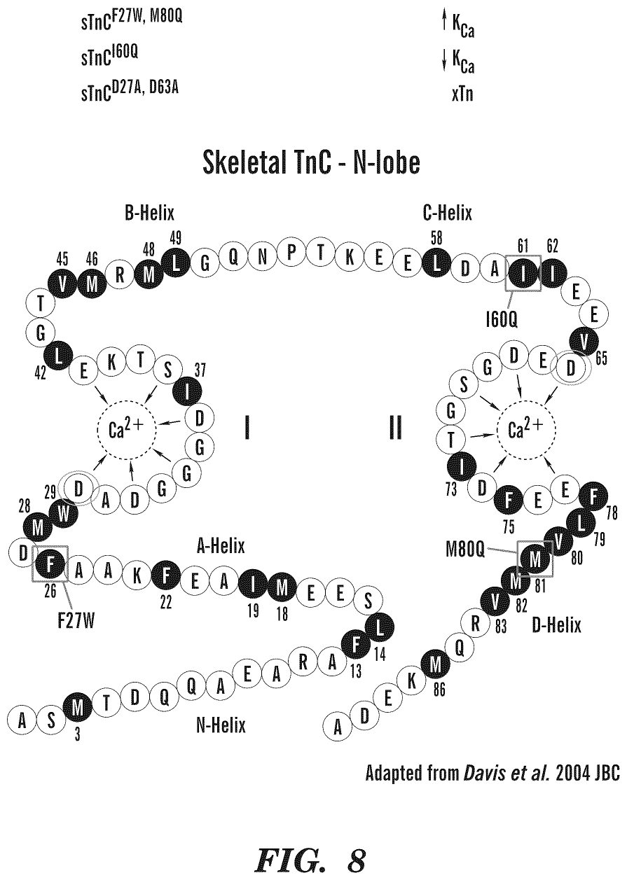

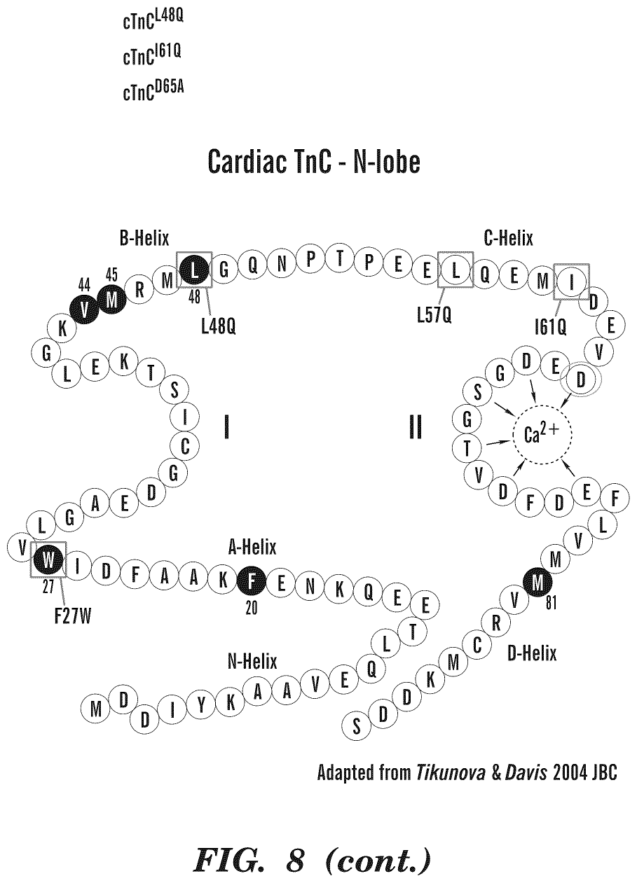

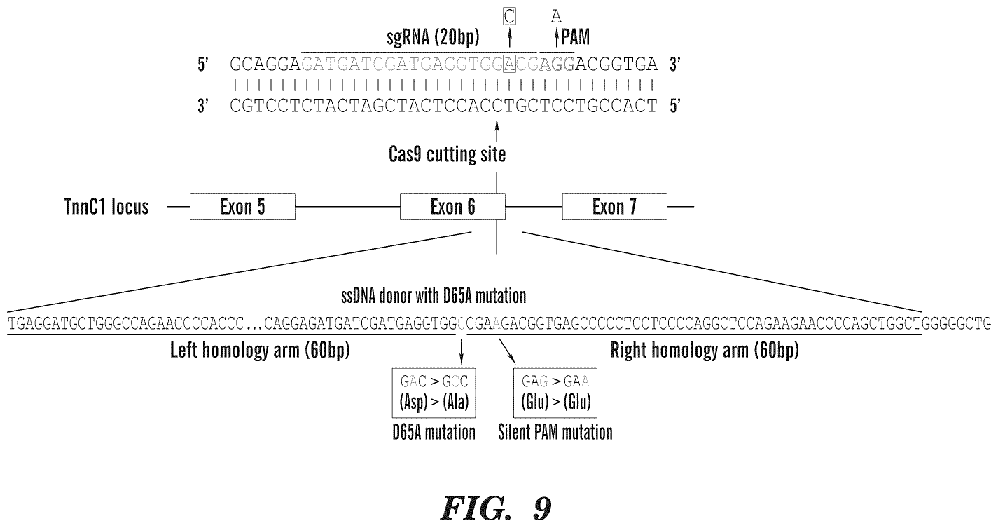

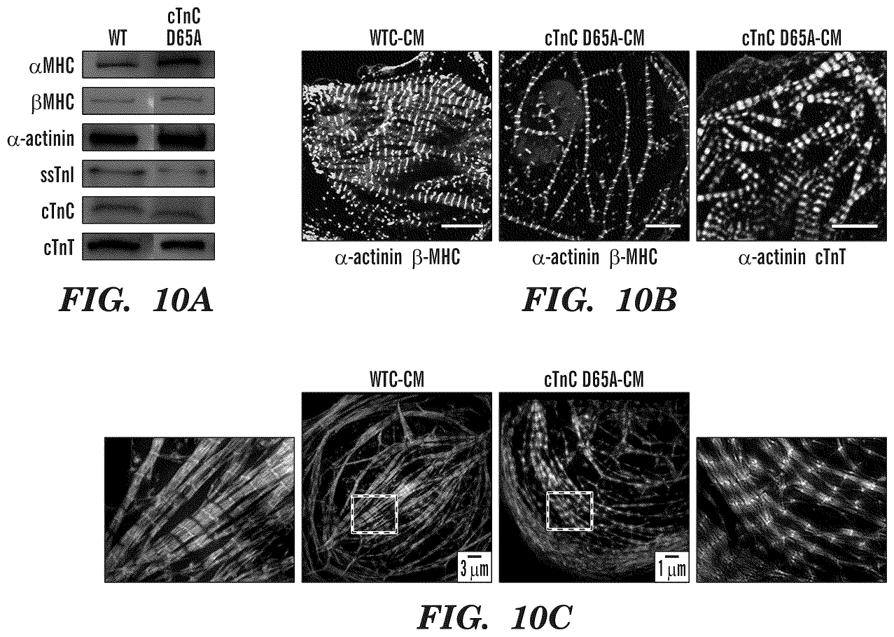

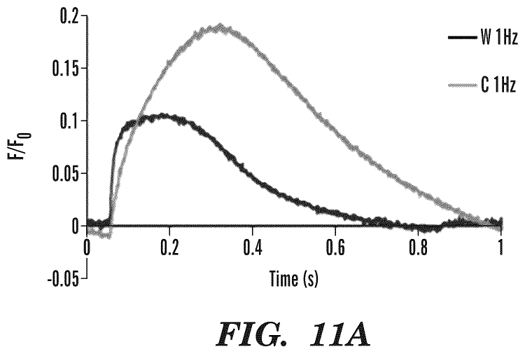

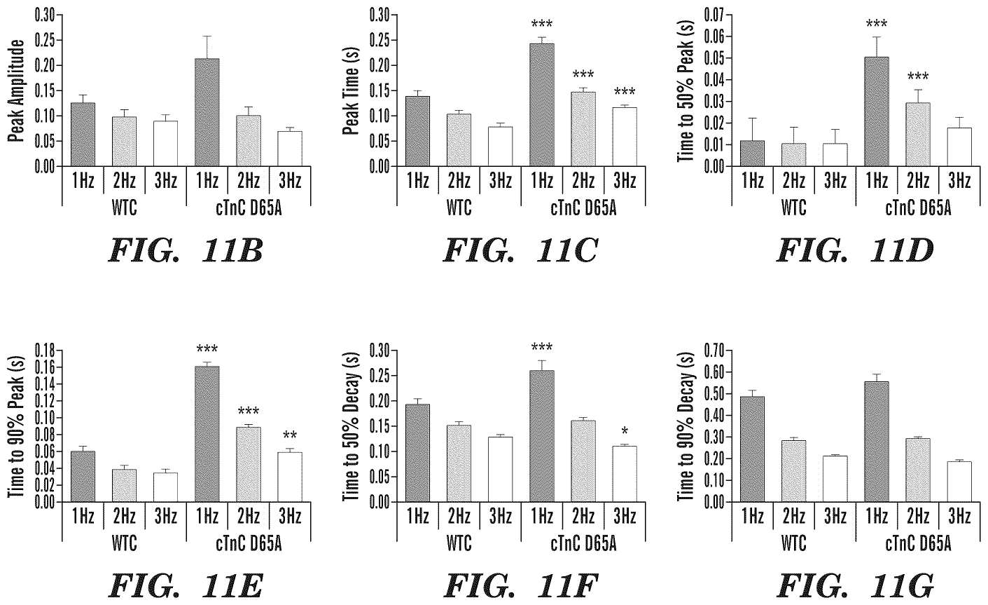

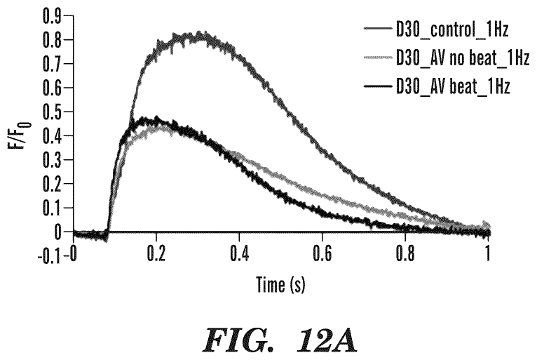

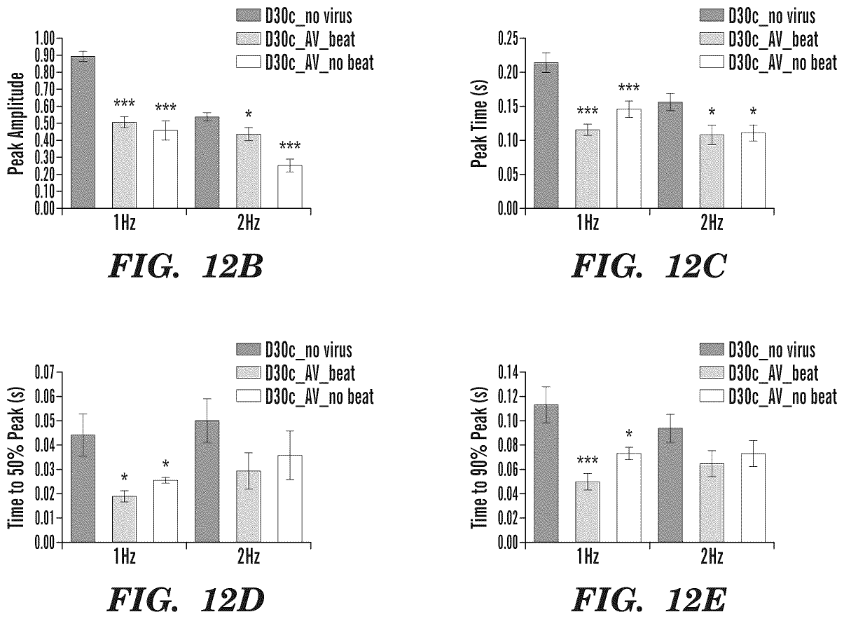

Human cardiomyocytes derived from ESCs or iPSCs can form stable grafts of new myocardium in injured hearts. These grafts prevent further decline in heart function in small animals (mice, rats, guinea pigs), and in non-human primates, they restore contractile function to near-normal levels. A longstanding question in the field is whether cardiomyocyte grafts work directly by adding new force-generating units, or whether passive mechanisms such as mechanically buttressing the infarcted wall or secreting paracrine factors are the predominant mechanism. The inventors have shown previously that human cardiomyocyte grafts electromechanically couple with the host myocardium and beat in synchrony, providing evidence in support of the direct contractile mechanism. To address this question more directly, the inventors generated human cardiomyocytes that were unable to contract, by knocking out key elements of the Ca2+-sensing troponin complex (TNNI1 and TNNI3) in human iPSCs. These iPSCs differentiated normally into cardiomyocytes, formed myofibrils with sarcomeres (albeit less robustly than wild type cells), and exhibited rhythmic Ca²⁺ transients. Importantly, these cells did not beat, indicating that excitation-contraction coupling had been interrupted successfully. The inventors then transplanted these cardiomyocytes into the infarcted hearts of athymic rats and compared their effects on contractile function to wild type, normally contractile cardiomyocytes. Surprisingly, the non-contractile TNNI1/3 knockout cardiomyocytes were equipotent with wild type cardiomyocytes in preventing the decline of systolic function after myocardial infarction (see, e.g., FIG. 6). Thus, in the rat, where human cardiomyocytes prevent functional deterioration without restoring systolic function, human cardiomyocytes have a non-contractile mechanism of action.

Similar experiments were performed in non-human primates (NHPs), where restoration of function has been observed. FIG. 7 shows data indicating cardiac recovery in control treated non-human primates (NIP), wild-type cardiomyocyte treated NHPs, and in an NHP treated with non-contractile cardiomyocytes. These data indicate that there is a comparable loss of ejection fraction in all NHPs post infarction. No spontaneous improvement was observed in NHPs receiving vehicle injection while a robust improvement was observed at 4 weeks in all NHPs receiving contractile cardiomyocytes, with further improvement out to 12 weeks. An equivalent improvement was observed at 4 weeks in the one NHP receiving non-contractile cardiomyocytes, and then minimal improvement from weeks 4-12; that is no further improvement occurred in the heart receiving non-contractile cardiomyocytes, but the improvement seen in the first four weeks was maintained. Thus, in NHPs, where function is restored, human cardiomyocytes can have a two-component mechanism of action, consisting of non-contractile and direct contractile benefits.

1. A transplant composition comprising a non-contractile cardiomyocyte, wherein the non-contractile cardiomyocyte exhibits impaired excitation-contraction coupling.

2. The transplant composition of claim 1, wherein the non-contractile cardiomyocyte is engineered to exhibit impaired excitation-contraction coupling.

3. The transplant composition of claim 1 or claim 2, wherein the non-contractile cardiomyocyte is a human non-contractile cardiomyocyte.

4. The transplant composition of any preceding claim, wherein the non-contractile cardiomyocyte exhibits normal action potentials.

5. The transplant composition of any preceding claim, wherein the non-contractile cardiomyocyte exhibits normal calcium transients.

6. The transplant composition of any preceding claim, wherein the non-contractile cardiomyocyte exhibits impaired function of a thin filament.

7. The transplant composition of any preceding claim, wherein the non-contractile cardiomyocyte is engineered to exhibit impaired function of a thin filament.

8. The transplant composition of any preceding claim, wherein impaired function of a thin filament comprises impaired calcium binding to troponin.

9. The transplant composition of any preceding claim, wherein impaired calcium binding to troponin is a result of knockout or mutation of at least one troponin isoform.

10. The transplant composition of any preceding claim, wherein the non-contractile cardiomyocyte comprises knockout of troponin I (TnI) and/or troponin T (TnT).

11. The transplant composition of any preceding claim, wherein the non-contractile cardiomyocyte comprises a mutation in troponin C that impairs calcium binding to troponin.

12. The transplant composition of claim 11, wherein the mutation in troponin C comprises D65A.

13. The transplant composition of claim 11, wherein the mutation in troponin C comprises I61Q, and/or L57Q.

14. The transplant composition of any preceding claim, wherein the non-contractile cardiomyocyte exhibits impaired actin-myosin binding.

15. The transplant composition of any preceding claim, wherein the non-contractile cardiomyocyte is engineered to have impaired actin-myosin binding.

16. The transplant composition of claim 14 or 15, wherein the impaired actin-myosin binding comprises a mutation in or deletion of myosin.

17. The transplant composition of any preceding claim, further comprising a pharmaceutically acceptable carrier.

18. The transplant composition of any preceding claim, wherein the non-contractile cardiomyocyte is derived from in vitro differentiation of a pluripotent stem cell or a cardiac progenitor cell.

19. The transplant composition of any preceding claim, wherein the non-contractile cardiomyocyte retains normal gap junction function when contacted with a cardiomyocyte.

20. A method for improving cardiac function in a subject in need thereof, the method comprising administering a cardiac transplant composition of any one of claim 1-19 to cardiac tissue of a subject in need thereof.

21. A method for improving cardiac function in a subject in need thereof, the method comprising: administering a non-contractile cardiomyocyte or a composition thereof to a graft site in cardiac tissue of a recipient in need thereof, thereby improving cardiac function in the subject.

22. The method of claim 21, wherein the non-contractile cardiomyocyte exhibits impaired excitation-contraction coupling.

23. The method of claim 21 or 22, wherein the non-contractile cardiomyocyte is engineered to exhibit impaired excitation-contraction coupling.

24. The method of any one of claims 21-23, wherein the non-contractile cardiomyocyte exhibits normal action potentials.

25. The method of claim any one of claims 21-24, wherein the non-contractile cardiomyocyte exhibits normal calcium transients.

26. The method of any one of claims 21-25, wherein the non-contractile cardiomyocyte exhibits impaired function of a thin filament.

27. The method of any one of claims 21-26, wherein the non-contractile cardiomyocyte is engineered to exhibit impaired function of a thin filament.

28. The method of claim 26 or 27, wherein the impaired function of a thin filament comprises impaired calcium binding to troponin.

29. The method of claim 28, wherein the impaired calcium binding to troponin is a result of knockout or mutation of at least one troponin isoform.

30. The method of any one of claims 22-29, wherein the non-contractile cardiomyocyte comprises knockout of troponin I (TnI) and/or troponin T (TnT).

31. The method of claim 28 or 29, wherein the non-contractile cardiomyocyte comprises a mutation in troponin C that impairs calcium binding to troponin.

32. The method of claim 31, wherein the mutation in troponin C comprises D65A.

33. The method of any one of claims 22-27, wherein the non-contractile cardiomyocyte exhibits impaired actin-myosin binding.

34. The method of any one of claims 22-27, wherein the non-contractile cardiomyocyte is engineered to exhibit impaired actin-myosin binding.

35. The method of claim 33 or 34, wherein the impaired actin-myosin binding comprises a mutation in or deletion of myosin.

36. The method of any one of claims 20-35, wherein the improvement in cardiac function comprises an increase in regional wall motion, fractional shortening, or ejection fraction.

37. The method of any one of claims 20-36, wherein the subject in need thereof comprises a cardiac disease or disorder.

38. The method of claim 37, wherein the cardiac disease or disorder comprises impaired contractility.

39. The method of claim 37 or 38, wherein the cardiac disease or disorder comprises a myocardial infarction, an ischemia/reperfusion injury, a cardiomyopathy or heart failure.

40. The method of any one of claims 20-39, wherein the risk or incidence of engraftment arrhythmia is reduced compared to a graft comprising wild-type cardiomyocytes.

41. The method of any one of claims 20-40, further comprising administering a wild-type cardiomyocyte in combination with the non-contractile cardiomyocyte.

42. A transplant composition comprising a cardiomyocyte that exhibits attenuated contractility and impaired excitation-contraction coupling.

43. A transplant composition comprising a cardiomyocyte engineered to have attenuated contractility and/or impaired excitation-contraction coupling.

44. The transplant composition of claim 42 or 43, wherein the cardiomyocyte is human.

45. The transplant composition of any one of claims 42-44, wherein the cardiomyocyte engineered to have attenuated contractility exhibits normal action potentials.

46. The transplant composition of any one of claims 42-45, wherein the cardiomyocyte engineered to have attenuated contractility exhibits normal calcium transients.

47. The transplant composition of any one of claims 42-46, wherein the non-contractile cardiomyocyte exhibits reduced function of a thin filament.

48. The transplant composition of any one of claims 42-47, wherein the non-contractile cardiomyocyte is engineered to exhibit reduced function of a thin filament.

49. The transplant composition of claim 47 or 48, wherein the reduced function of a thin filament comprises reduced calcium binding to troponin.

50. The transplant composition of claim 49, wherein the reduced calcium binding to troponin is a result of a mutation in troponin C that reduces calcium binding to troponin C by at least 20%.

51. The transplant composition of claim 50, wherein the mutation in troponin C comprises I61Q and/or L57Q.

52. The transplant composition of any one of claims 42-48, wherein the cardiomyocyte comprises reduced actin-myosin binding relative to wild-type.

53. The transplant composition of any one of claims 42-52, further comprising a pharmaceutically acceptable carrier.

54. The transplant composition of any one of claims 42-53, wherein the cardiomyocyte that has reduced contractility is derived from in vitro differentiation of a pluripotent stem cell or a cardiac progenitor cell.

55. The transplant composition of any one of claims 42-54, wherein the cardiomyocyte having reduced contractility retains normal gap junction function when contacted with a cardiomyocyte.

56. A method for improving cardiac function in a subject in need thereof, the method comprising administering a transplant composition of any one of claims 42-55 to cardiac tissue of a subject in need thereof.

57. A method for improving cardiac function in a subject in need thereof, the method comprising: administering a cardiomyocyte that exhibits attenuated contractility or a composition thereof to a graft site in cardiac tissue of the subject in need thereof, thereby improving cardiac function in the subject.

58. The method of claim 56 or 57, wherein the improvement in cardiac function comprises an increase in regional wall motion, fractional shortening, or ejection fraction.

59. The method of claim any one of claims 56-58, wherein the subject in need thereof comprises a cardiac disease or disorder.

60. The method of claim 59, wherein the cardiac disease or disorder comprises reduced contractility.

61. The method of claim 60, wherein the cardiac disease or disorder comprises a myocardial infarction, an ischemia/reperfusion injury, a cardiomyopathy or heart failure.

62. The method of any one of claims 56-61, wherein the risk or incidence of engraftment arrhythmia is reduced compared to a graft comprising wild-type cardiomyocytes.

63. The method of any one of claims 56-61, further comprising administering a wild-type cardiomyocyte in combination with the cardiomyocyte engineered to have attenuated contractility.

64. A unit dosage formulation comprising non-contractile cardiomyocytes for administration to cardiac tissue of a subject in need thereof, the unit dosage formulation comprising 1×10³ to 5×10⁹ non-contractile cardiomyocytes in a gel or matrix.

65. The unit dosage formulation of claim 64, wherein the non-contractile cardiomyocyte is engineered to exhibit impaired excitation-contraction coupling.

66. The unit dosage formulation of claim 64 or claim 65, wherein the non-contractile cardiomyocyte is a human non-contractile cardiomyocyte.

67. The unit dosage formulation of any one of claims 64-66, wherein the non-contractile cardiomyocyte exhibits normal action potentials.

68. The unit dosage formulation of any one of claims 64-67, wherein the non-contractile cardiomyocyte exhibits normal calcium transients.

69. The unit dosage formulation of any one of claims 64-68, wherein the non-contractile cardiomyocyte exhibits impaired function of a thin filament.

70. The unit dosage formulation of claim 69, wherein the non-contractile cardiomyocyte is engineered to exhibit impaired function of a thin filament.

71. The unit dosage formulation of claim 69 or 70, wherein impaired function of a thin filament comprises impaired calcium binding to troponin.

72. The unit dosage formulation of claim 71, wherein calcium binding to troponin is a result of knockout or mutation of at least one troponin isoform.

73. The unit dosage formulation of claim 72, wherein the non-contractile cardiomyocyte comprises knockout of troponin I (TnI) and/or troponin T (TnT).

74. The unit dosage formulation of claim 72, wherein the non-contractile cardiomyocyte comprises a mutation in troponin C that impairs calcium binding to troponin.

75. The unit dosage formulation of claim 72 or 74 wherein the mutation comprises D65A mutation of troponin C.

76. The unit dosage formulation of claim 72 or 74, wherein the mutation comprises I61Q and/or L57Q mutation of troponin C.

77. The unit dosage formulation of any one of claims 64-76, wherein the non-contractile cardiomyocyte exhibits impaired actin-myosin binding.

78. The unit dosage formulation of any one of claims 64-77, wherein the non-contractile cardiomyocyte is engineered to have impaired actin-myosin binding.

79. The unit dosage formulation of claim 77 or 78, wherein the impaired actin-myosin binding comprises a mutation in or deletion of myosin.

80. The unit dosage formulation of any one of claims 64-79, further comprising a pharmaceutically acceptable carrier.

81. The unit dosage formulation of any one of claims 64-80, wherein the non-contractile cardiomyocyte is derived from in vitro differentiation of a pluripotent stem cell or a cardiac progenitor cell.

82. The unit dosage formulation of any one of claims 64-81, wherein the non-contractile cardiomyocyte retains normal gap junction function when contacted with a cardiomyocyte.

83. The unit dosage formulation of any one of claims 64-82, wherein the gel or matrix comprises a solubilized basement membrane protein or preparation thereof.

84. The unit dosage formulation of any one of claims 64-83, wherein the formulation further comprises one or more of an immunosuppressive agent, a pan-caspase inhibitor, an anti-apoptotic agent, IGF-1 and a KATP channel opening agent.

85. The unit dosage formulation of any one of claims 64-84, wherein the unit dosage formulation comprises 1×10³ to 5×10⁹ non-contractile cardiomyocytes.

86. The unit dosage formulation of any one of claims 64-85, wherein the unit dosage formulation comprises 1×10⁴ to 5×10⁹ non-contractile cardiomyocytes.

87. The unit dosage formulation of any one of claims 64-86, wherein the unit dosage formulation comprises 1×10⁵ to 5×10⁹ non-contractile cardiomyocytes.

88. The unit dosage formulation of any one of claims 64-68, wherein the unit dosage formulation comprises 1×10⁶ to 5×10⁹ non-contractile cardiomyocytes.

89. The unit dosage formulation of any one of claims 64-88, wherein the unit dosage formulation comprises 1×10⁷ to 5×10⁹ non-contractile cardiomyocytes.

90. The unit dosage formulation of any one of claims 64-89, wherein the unit dosage formulation comprises 1×10⁸ to 5×10⁹ non-contractile cardiomyocytes.

91. The unit dosage formulation of any one of claims 64-90, wherein the unit dosage formulation comprises 1×10⁵ to 1×10⁹ non-contractile cardiomyocytes.

92. The unit dosage formulation of any one of claims 64-91, wherein the unit dosage formulation comprises 1×10⁵ to 5×10⁸ non-contractile cardiomyocytes.

93. The unit dosage formulation of any one of claims 64-92, wherein the unit dosage formulation comprises 1×10⁵ to 1×10⁸ non-contractile cardiomyocytes.

94. The unit dosage formulation of any one of claims 64-93, wherein the unit dosage formulation comprises 1×10⁵ to 5×10⁷ non-contractile cardiomyocytes.

95. The unit dosage formulation of any one of claims 64-94, wherein the unit dosage formulation comprises 1×10⁵ to 1×10⁷ non-contractile cardiomyocytes.

96. The unit dosage formulation of any one of claims 64-95, wherein the unit dosage formulation comprises 1×10⁵ to 5×10⁶ non-contractile cardiomyocytes.

97. The unit dosage formulation of any one of claims 66-96, wherein the unit dosage formulation comprises 1×10⁵ to 1×10⁶ non-contractile cardiomyocytes.
